(19) United States
(12) Patent Application Publication
(10) Pub. No.: US 2022/0202799 A1
(43) Pub. Date: Jun. 30, 2022

(54) PHARMACEUTICAL FORMULATIONS CONTAINING P2Y14 ANTAGONISTS

(71) Applicant: KANTUM PHARMA, INC., Concord, NH (US)

(72) Inventors: John Randle, Boston, MA (US); Yadan Chen, Concord, NH (US)

(21) Appl. No.: 17/601,855

(22) PCT Filed: Apr. 13, 2020
(86) PCT No.: PCT/US2020/027882

§ 371 Date: Oct. 6, 2021

Related U.S. Application Data
(60) Provisional application No. 62/834,517, filed on Apr. 16, 2019.

(51) Int. Cl. A61K 31/451; A61K 47/14; A61K 47/40; A61P 13/12

(52) U.S. Cl. CPC A61K 31/451; A61P 13/12; A61K 47/40; A61K 47/14

(57) ABSTRACT

The invention provides formulations containing a P2Y14 antagonist, such as the naphthoic acid derivative PPTN, and an agent that increases the solubility of the P2Y 14 antagonist in an aqueous medium. The invention also provides methods of using the formulations to treat kidney disorders and conditions associated with renal inflammation.

### CROSS-REFERENCE TO RELATED APPLICATIONS

This application claims the benefit of, and priority to, U.S. Provisional Patent Application No. 62/834,517, filed Apr. 16, 2019, the contents of which are incorporated by reference.

### FIELD OF THE INVENTION

The invention relates generally to pharmaceutical formulations that contain P2Y14 antagonists, such as naphthoic acid derivatives.

### BACKGROUND

Nephritis, or inflammation of the kidneys, is among the top ten causes of death for women in the United States. Inflammation of the kidneys impairs their ability to filter blood, so the blood accumulates metabolic waste products and loses essential components, either of which may be fatal. For example, the loss of factors that limit blood clotting may result in a stroke. Nephritis may be triggered by a variety of conditions, such as cardiac surgery, kidney transplantation, infections, diabetes, hypertension, injuries, and autoimmune disorders such as lupus.

One compound that has shown promise as a therapeutic agent to limit renal inflammation is the naphthoic acid derivative 4-((piperidin-4-yl)-phenyl)-(7-(4-(trifluoromethyl)-phenyl)-2-naphthoic acid (PPTN). PPTN functions by inhibiting the purinergic receptor P2Y14, a key mediator of renal inflammation. However, PPTN is highly hydrophobic and has low water-solubility, so it has low bioavailability when administered to patients as a pharmacological agent. Efforts to overcome these problems by making chemical modifications of PPTN have failed because the altered compounds are inactive toward P2Y14. Consequently, current treatments for nephritis are inadequate, and the condition continues to lead to death or serious medical problems for millions of people each year.

### SUMMARY

The invention provides pharmaceutical formulations containing solutions of P2Y14 antagonists, such as PPTN, at therapeutically useful concentrations. The formulations include a non-toxic agent, such as sulfobutyl ether beta-cyclodextrin (SBECD) or α-tocopherol polyethylene glycol succinate (TPGS), that increases the solubility of naphthoic acid derivatives in aqueous solutions within a physiologically acceptable pH range, e.g., 5.0-8.0. Consequently, the formulations allow direct administration, e.g., by intravenous injection or infusion, of therapeutically effective quantities of naphthoic acid derivatives such as PPTN or other P2Y14 antagonists.

Because the formulations of the invention allow delivery of PPTN and other P2Y14 antagonists at high levels, the formulations are useful for the treatment or prevention of renal inflammation. Thus, the invention also provides methods of treating or preventing renal inflammation in a subject using the formulations described herein.

In an aspect, the invention provides pharmaceutical formulations that contain a P2Y14 antagonist and an agent.

The P2Y14 antagonist may be a naphthoic acid derivative or salt thereof. The naphthoic acid derivative or salt thereof may be 4-((piperidin-4-yl)-phenyl)-(7-(4-(trifluoromethyl)-phenyl)-2-naphthoic acid (PPTN). The salt of the naphthoic acid derivative may be PPTN hydrochloride. The P2Y14 antagonist may be a triazole derivative.

The formulation may contain the P2Y14 antagonist at a certain concentration. For example, the P2Y14 antagonist may be present in the formulation at ≥0.001 μg/ml, ≥0.002 μg/ml, ≥0.005 μg/ml, ≥0.01 μg/ml, ≥0.02 μg/ml, ≥0.05 μg/ml, ≥0.1 μg/ml, ≥0.2 μg/ml, ≥0.5 μg/ml, >1 μg/ml, ≥2 μg/ml, >5 μg/ml, >10 μg/ml, >20 μg/ml, >50 μg/ml, >100 μg/ml, >200 μg/ml, >500 μg/ml, >1 mg/ml, >2 mg/ml, >5 mg/ml, or ≥10 mg/ml. The P2Y14 antagonist may be present in the formulation at from about 1 μg/ml to about 20 mg/ml, from about 2 μg/ml to about 20 mg/ml, from about 5 μg/ml to about 20 mg/ml, from about 10 μg/ml to about 20 mg/ml, from about 20 μg/ml to about 20 mg/ml, from about 50 μg/ml to about 20 mg/ml, from about 100 μg/ml to about 20 mg/ml, from about 200 μg/ml to about 20 mg/ml, from about 500 μg/ml to about 20 mg/ml, from about 1 mg/ml to about 20 mg/ml, from about 2 mg/ml to about 20 mg/ml, from about 5 mg/ml to about 20 mg/ml, from about 1 μg/ml to about 10 mg/ml, from about 2 μg/ml to about 10 mg/ml, from about 5 μg/ml to about 10 mg/ml, from about 10 μg/ml to about 10 mg/ml, from about 20 μg/ml to about 10 mg/ml, from about 50 μg/ml to about 10 mg/ml, from about 100 μg/ml to about 10 mg/ml, from about 200 μg/ml to about 10 mg/ml, from about 500 μg/ml to about 10 mg/ml, from about 1 mg/ml to about 10 mg/ml, from about 2 mg/ml to about 10 mg/ml, from about 5 mg/ml to about 10 mg/ml, from about 1 μg/ml to about 5 mg/ml, from about 2 μg/ml to about 5 mg/ml, from about 5 μg/ml to about 5 mg/ml, from about 10 μg/ml to about 5 mg/ml, from about 20 μg/ml to about 5 mg/ml, from about 50 μg/ml to about 5 mg/ml, from about 100 μg/ml to about 5 mg/ml, from about 200 μg/ml to about 5 mg/ml, from about 500 μg/ml to about 5 mg/ml, from about 1 mg/ml to about 5 mg/ml, from about 2 mg/ml to about 5 mg/ml, from about 1 μg/ml to about 2 mg/ml, from about 2 μg/ml to about 2 mg/ml, from about 5 μg/ml to about 2 mg/ml, from about 10 μg/ml to about 2 mg/ml, from about 20 μg/ml to about 2 mg/ml, from about 50 μg/ml to about 2 mg/ml, from about 100 μg/ml to about 2 mg/ml, from about 200 μg/ml to about 2 mg/ml, from about 500 μg/ml to about 2 mg/ml, or from about 1 mg/ml to about 2 mg/ml.

The pharmaceutical formulation may be an aqueous solution.

The agent may increase to the solubility of the P2Y14 antagonist in an aqueous solution. The agent may be α-tocopherol polyethylene glycol succinate (TPGS) or sulfobutyl ether beta-cyclodextrin (SBECD).

The formulation may contain the agent at a certain concentration. For example, the agent may be present in the formulation at less than about 40%, less than about 35%, less than about 30%, less than about 25%, less than about 20%, less than about 15%, less than about 10%, less than about 5%, less than about 2%, less than about 1%, less than about 0.5%, less than about 0.2%, less than about 0.1%, less than about 0.05%, less than about 0.02%, less than about 0.01%, less than about 0.005%, less than about 0.002%, or less than about 0.001%. The agent may be present in the formulation at from about 0.001% to about 0.01%, from about 0.003% to about 0.03%, from about 0.01% to about 0.1%, from about 0.03% to about 0.03%, from about 0.1% to about 1%, from about 0.3% to about 3%, from about 1% to about 10%, from about 2% to about 10%, from about 3% to about 10%, from about 5% to about 10%, from about 5% to about 12%, from about 5% to about 15%, from about 5% to about 20%, from about 7.5% to about 10%, from about 7.5% to about 12%, from about 7.5% to about 15%, from about 7.5% to about 20%, from about 10% to about 12%, from about 10% to about 15%, or from about 10% to about 20%.

The formulation may have a pH in a physiologically-compatible range. The formulation may have a pH of ≥4.0, ≥4.5, ≥5.0, ≥5.5, ≥6.0, ≥6.5, ≥7.0, ≥7.5, or ≥8.0. The formulation may have a pH within a range. For example, the formulation may have a pH of from about 4.0 to about 9.0, from about 5.0 to about 9.0, from about 6.0 to about 9.0, from about 7.0 to about 9.0, from about 4.0 to about 8.0, from about 5.0 to about 8.0, from about 6.0 to about 8.0, from about 7.0 to about 8.0, from about 4.0 to about 7.0, from about 5.0 to about 7.0, or from about 6.0 to about 7.0. The formulation may have a pH of about 5.0, about 5.5, about 6.0, about 6.5, about 7.0, about 7.5, or about 8.0.

The formulation may contain a buffering agent and/or one or more salts. The buffering agent may be phosphate. The salt may be sodium chloride or potassium chloride. The formulation may contain saline or phosphate-buffered saline.

The formulation may contain dimethyl sulfoxide (DMSO). DMSO may be present in the formulation at less than about 10%, less than about 5%, less than about 3%, less than about 2%, less than about 1%, less than about 0.5%, less than about 0.3%, less than about 0.2%, or less than about 0.1%.

The formulation may be substantially free of solvents or other chemicals that may be toxic to a subject. For example, the formulation may be substantially free of dimethylacetamide (DMAc), ethanol, N-methylpyrrolidone (NMP), and/or polyethylene glycol (PEG).

In another aspect, the invention provides methods of treating a renal disorder by providing to a subject a formulation containing a P2Y14 antagonist and an agent.

The renal disorder may be any disease, disorder, or condition associated with renal inflammation. The renal disorder or condition associated with renal inflammation may be accumulation of mesangial matrix, acute interstitial nephritis, acute kidney injury (AKI), including AKI associated with cardiac surgery, acute tubular necrosis (ATN), Alport syndrome, atherosclerosis, atherosclerotic renal artery stenosis, an autoimmune disorder, such us systemic lupus erythematosus, autosomal dominant polycystic kidney disease (ADPKD), benign prostatic hyperplasia, bladder stones, cancer, including cancer of the bladder, ureters, or prostate, cardiac surgery, cell apoptosis, chronic kidney disease, chronic tubulointerstitial nephritis, delayed graft function (DGF), including DGF-renal, diabetes, diabetic nephropathy, including type 1 diabetic nephropathy (T1D nephropathy), end-stage renal disease, Focal segmental glomerulosclerosis (FSGS), glomerular basement membrane thickening, glomerular hyperfiltration, glomerular and tubular epithelial hypertrophy, glomerulonephritis, glomerulosclerosis, heart failure, hemolytic-uremic syndrome, hypertension, IgA nephropathy (also called Berger's disease), infection, injury, ischemia, ischemia/reperfusion injury, ischemic nephropathy, kidney hypoxia, kidney stones, kidney transplantation, liver cirrhosis, methyl melonic acidosis (MMA), microalbuminuria, obstructed urinary catheter, proteinuria, reduced creatinine clearance, reduced glomerular filtration rate, reflux nephropathy, renal vein thrombosis, rhabdomyolysis, tumor lysis syndrome, vascular occlusion, or vasculitis.

The formulation may have any of the properties described above in relation to formulations of the invention. For example, the P2Y14 antagonist may be a naphthoic acid derivative or salt thereof, and the agent may be SBECD or TPGS.

### BRIEF DESCRIPTION OF THE DRAWINGS

FIG. 1 is graph showing HPLC analysis of a single concentration of PPTN.

FIG. 2 is graph showing HPLC analysis of different concentrations of PPTN.

FIG. 3 is a graph of UV absorption at 268 nm vs. concentration of PPTN.

FIG. 4 is graph showing the concentration of PPTN in saturated solutions that contain different amounts of SBECD.

FIG. 5 is a graph of PPTN concentrations of samples tested in stability assay.

FIG. 6 is a representative UHPLC-UV trace for the sample at 40° C., time=28 days.

FIG. 7 is a graph of plasma PPTN concentration over time in rats after a single bolus injection.

FIG. 8 is a graph of plasma PPTN concentration as a function of dose in rats after a single bolus injection.

FIG. 9 is a graph of urine PPTN concentration over time in rats after a single bolus injection.

FIG. 10 is a graph of plasma PPTN concentration over time in rats during continuous intravenous administration for 24 hours.

FIG. 11 is a graph of plasma PPTN concentration over time in rats during continuous intravenous administration for 72 hours.

FIG. 12 is a graph of plasma PPTN concentration as a function of dose in rats during continuous intravenous infusion.

FIG. 13 is a standard curve of plasma PPTN concentrations from rats that received continuous intravenous infusion at 10 mg/kg for 24 or 72 hours.

FIG. 14 is a standard curve of plasma PPTN concentrations from rats that received continuous intravenous infusion at 30 mg/kg for 24 or 72 hours.

FIG. 15 is a standard curve of plasma PPTN concentrations from rats that received continuous intravenous infusion at 100 mg/kg for 24 or 72 hours.

FIG. 16 is a standard curve of urine PPTN concentrations from rats that received continuous intravenous infusion at 10 mg/kg for 24 or 72 hours.

FIG. 17 is a standard curve of urine PPTN concentrations from rats that received continuous intravenous infusion at 30 mg/kg for 24 or 72 hours.

FIG. 18 is a standard curve of urine PPTN concentrations from rats that received continuous intravenous infusion at 100 mg/kg for 24 or 72 hours.

FIG. 19 is a standard curve of a summary of urine concentrations of PPTN in rats that received continuous intravenous infusion for 24 or 72 hours.

### DETAILED DESCRIPTION

Renal inflammation, or nephritis, is potentially life-threating condition associated with a wide range of diseases, disorders, and conditions. For example, renal inflammation may result from cardiac surgery, kidney transplantation, infections, diabetes, hypertension, injuries, and autoimmune disorders, such as lupus. Inflammation impairs the kidneys' ability to filter blood, resulting in the accumulation of waste products and the loss of vital blood components.

Recent reports have identified the purinergic receptor P2Y14, also called GPR105, as a key mediator of renal inflammation. The gene and protein for human P2Y14 are described in, for example, Entrez Gene ID no. 9934, GenBank ID no. D13626, RefSeq ID no. NM_014879, and UniProt ID no. NM_01487, the contents of which are incorporated herein by reference. P2Y14 is a G protein-coupled receptor expressed on the surface of intercalated cells (ICs) in the collecting duct system of the kidney. P2Y14 binds uridine diphosphate glucose (UDP-glucose), an ester of pyrophosphoric acid with the nucleoside uridine, and related UDP-hexoses, such as UDP-galactose, UDP-glucuronic acid, N-acetyl-UDP-glucosamine and N-acetyl-UDP-galactosamine. Abbracchio et al., Characterization of the UDP-glucose receptor (re-named here the P2Y14 receptor) adds diversity to the P2Y receptor family, Trends Pharmacol Sci. 2003 February; 24(2):52-5, DOI: 10.1016/S0165-6147(02)00038-X, the contents of which are incorporated herein by reference. These hexoses are agonists for the P2Y14 receptor, and throughout this text, “UDP-glucose” and “UDP-glucose and related UDP-hexoses” refer to this group of hexoses unless otherwise expressly indicated that only UDP-glucose is intended. UDP-glucose and related UDP-hexoses are released into extracellular fluids from damaged cells and in a regulated manner from intact cells. Binding of UDP-glucose to P2Y14 triggers ICs to produce chemokines that lead to infiltration of neutrophils into the renal medulla. See Azroyan et al., Renal Intercalated Cells Sense and Mediate Inflammation via the P2Y14 Receptor, PLoS ONE 10(3): e0121419 (2015), doi:10.1371/joumal.pone.0121419. Thus, high levels of circulating UDP-glucose activate P2Y14 to cause renal inflammation.

The naphthoic acid derivative 4-((piperidin-4-yl)-phenyl)-(7-(4-(trifluoromethyl)-phenyl)-2-naphthoic acid (PPTN) has been identified as an antagonist of P2Y14. However, PPTN is highly hydrophobic, which presents challenges in developing formulations that deliver PPTN at levels sufficient to combat renal inflammation. Prior formulations of PPTN have poor bioavailability. The solubility of PPTN is increased under acidic conditions, but highly acidic solutions disrupt the pH of the blood when administered intravenously and thus are not suitable for therapeutic use. In addition, efforts to modify the molecule to increase its water-solubility have failed because the PPTN derivatives are poor antagonists of P2Y14.

The invention overcomes the aforementioned problems by providing formulations that contain P2Y14 antagonists, including naphthoic acid derivatives such as PPTN, at concentrations sufficient to provide therapeutic benefit. The formulations include one or more agents, such as sulfobutyl ether beta-cyclodextrin (SBECD) and α-tocopherol polyethylene glycol succinate (TPGS), that increase the solubility of PPTN and similar compounds at near-neutral pH. Consequently, the formulations allow delivery of high levels of P2Y14 antagonists to treat renal inflammation without disrupting the body's homeostatic processes.

### Formulations

P2Y14 Antagonists

The invention provides formulations that contain P2Y14 antagonists. The P2Y14 antagonist may be any entity that interferes with ligand-binding, activation, or signaling by P2Y14. The P2Y14 antagonist may be a small or large organic or inorganic molecule. The P2Y14 antagonist may be a 4,7-disubstituted naphthoic acid derivative, such as one of the compounds described in U.S. Publication No. 2010/0298347, the contents of which are incorporated herein by reference. Such compounds may be represented by formula (I):

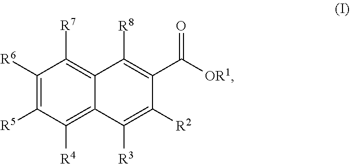

wherein:

R¹ is selected from the group consisting of hydrogen, C₃₋₆ cycloalkyl, benzyl, and C₁₋₆ alkyl wherein alkyl is optionally substituted with hydroxy, amino, C₁₋₄ alkylamino, di-(C₁₋₄ alkyl)amino, aminocarbonyl, C₁₋₄ alkylaminocarbonyl, di-(C₁₋₄-alkyl)aminocarbonyl, C₁₋₄ alkylcarbonyloxy, C₁₋₄ alkyloxy, or one to five fluorines;

R² is hydrogen, fluorine, or hydroxy;

R³ is selected from the group consisting of: —(CH₂)ₘ aryl, —(CH₂)ₘheteroaryl, —OCH₂-aryl, —OCH₂-heteroaryl, —(S)ᵣCH₂-aryl, —(S)ᵣCH₂-heteroaryl, —CH₂O-aryl, —CH₂O-heteroaryl, —CH₂(S)ᵣ-aryl, and —CH₂(S)ᵣ-heteroaryl;

wherein any methylene (CH₂) carbon atom in R3 is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group; and wherein aryl and heteroaryl are optionally substituted with one to three R^{c} substituents independently selected from the group consisting of:

halogen,

cyano,

nitro,

C₁₋₆ alkoxy, wherein alkoxy is optionally substituted with one to five substituents independently selected from fluorine, hydroxy, and C₁₋₃ alkoxy,

C₁₋₆ alkyl, wherein alkyl is optionally substituted with one to five substituents independently selected from fluorine, hydroxy, and C₁₋₃ alkoxy,

C₂₋₆ alkenyl, wherein alkenyl is optionally substituted with one to five substituents independently selected from fluorine, hydroxy, and C₁₋₃ alkoxy,

(CH₂)ₙ-aryl,

(CH₂)ₙ-heteroaryl,

(CH₂)ₙ-heterocyclyl,

(CH₂)ₙ—C₃₋₆ cycloalkyl,

(CH₂)ₙ—OR⁹,

(CH₂)ₙ—CO₂R⁹,

(CH₂)ₙ—N(R⁹)₂,

(CH₂)ₙ—CON(R⁹)₂,

(CH₂)ₙ—OCON(R⁹)₂,

(CH₂)ₙ—SO₂N(R⁹)₂,

(CH₂)ₙ—SO₂N(R⁹)C(O)R⁹,

(CH₂)ₙ—C(O)ₙ(R⁹)SO₂R¹⁰,

(CH₂)ₙ—S(O)ᵣR¹⁰,

(CH₂)ₙ—NR¹¹SO₂R¹⁰;

(CH₂)ₙ—NR¹¹CON(R⁹)₂,

(CH₂)ₙ—NR¹¹COR⁹, and

(CH₂)ₙ—NR¹¹CO₂R¹⁰;

wherein aryl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R^{c} is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group;

R⁴, R⁵, R⁷, and R⁸ are each independently selected from the group consisting of:

hydrogen,

halogen,

C₁₋₄ alkyl, optionally substituted with one to five fluorines,

C₁₋₄ alkoxy, optionally substituted with one to five fluorines, and

C₁₋₄ alkylthio, optionally substituted with one to five fluorines;

R6 is selected from the group consisting of:

—(CH₂)ₘ-aryl,

—(CH₂)ₘ-heteroaryl,

—OCH₂-aryl,

—OCH₂-heteroaryl,

—(S)ᵣCH₂-aryl,

—(S)ᵣCH₂-heteroaryl,

—CH₂O-aryl,

—CH₂O-heteroaryl,

—CH₂(S)ᵣ-aryl, and

—CH₂(S)ᵣ-heteroaryl;

wherein any methylene (CH₂) carbon atom in R6 is optionally substituted with one to two groups independently selected from fluorine, hydroxy, and C₁₋₄ alkyl optionally substituted with one to three fluorines; or two substituents when on the same methylene (CH₂) group are taken together with the carbon atom to which they are attached to form a cyclopropyl group and wherein aryl and heteroaryl are optionally substituted with one to three Rd substituents independently selected from the group consisting of:

halogen,

cyano,

C₁₋₄ alkyl, optionally substituted with one to five fluorines,

C₁₋₄ alkoxy, optionally substituted with one to five fluorines,

C₁₋₄ alkylthio, optionally substituted with one to five fluorines, and

C₁₋₄ alkylsulfonyl, optionally substituted with one to five fluorines;

each R⁹ is independently selected from the group consisting of hydrogen,

C₁, alkyl,

(CH₂)ₘ-aryl,

(CH₂)ₘ-heteroaryl, and

(CH₂)ₘC₃₋₆ cycloalkyl;

wherein any individual methylene (CH₂) carbon atom in (CH₂)ₘ is optionally substituted with one to two substituents independently selected from fluorine, hydroxy, C₁₋₄ alkyl, and C₁₋₄ alkoxy, wherein alkyl and alkoxy are optionally substituted with one to five fluorines; or two substituents when on the same methylene (CH₂) group are taken together with the carbon atom to which they are attached to form a cyclopropyl group; and wherein alkyl, aryl, heteroaryl, and cycloalkyl are optionally substituted with one to three substituents independently selected from the group consisting of halogen, C₁₋₄ alkyl, and C₁₋₄ alkoxy; or two R⁹ groups substituents together with the nitrogen atom to which they are attached form a heterocyclic ring selected from azetidine, pyrrolidine, piperidine, piperazine, and morpholine wherein said heterocyclic ring is optionally substituted with one to three substituents independently selected from the group consisting of halogen, hydroxy, C₁₋₆alkyl, and C₁₋₆ alkoxy, wherein alkyl and alkoxy are optionally substituted with one to five fluorines;
each R¹⁰ is independently C₁₋₆ alkyl, wherein alkyl is optionally substituted with one to five substituents independently selected from fluorine and hydroxy;
R¹¹ is hydrogen or R¹⁰;
each n is independently an integer from 0 to 3;
each m is independently an integer from 0 to 2; and
each r is an integer from 0 to 2.

The naphthoic acid derivative may be 4-[4-(piperidin-4-yl)phenyl]-7-[4-(trifluoromethyl)phenyl]-2-naphthoic acid (PPTN), which has the following structure:

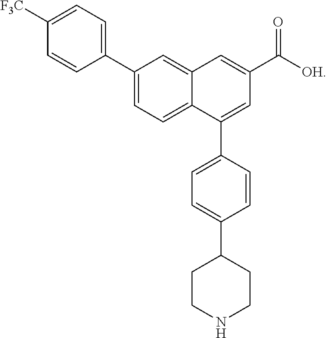 [SMILES: O=C(O)c1cc(-c2ccc(C3CCNCC3)cc2)c2ccc(-c3ccc(C(F)(F)F)cc3)cc2c1]

The P2Y14 antagonist may be a triazole derivative, such as one of the compounds described in WO 2017/053769, the contents of which are incorporated herein by reference. Such compounds may be represented by the formula (XI):

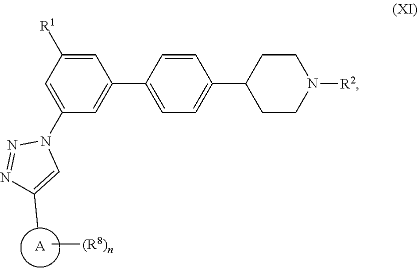

wherein

ring A is aryl, heteroaryl, or cycloalkyl;

R¹ is —CO₂H, —CO₂(C₁-C₈ alkyl), or a bioisostere of carboxylate;

R² is H, C₁-C₈ alkyl, C₂-C₈ alkenyl, C₂-C₈ alkynyl, C₃-C₆ cycloalkyl, C₃-C₆ cycloalkylalkyl, hydroxyalkyl, C₁-C₈ haloalkyl, cyanoalkyl, aryl, heteroaryl, heterocycloalkyl, —(CH₂)ₘaryl, —(CH₂)ₘheteroaryl, or —(CH₂)ₘheterocycloalkyl;

each R³ is the same or different and each is C₁-C₈ alkyl, C₂-C₈ alkenyl, C₃-C₆ cycloalkyl, hydroxy, hydroxyalkyl, C₁-C₈ alkoxy, C₃-C₆ cycloalkyloxy, aryloxy, halo, C₁-C₈ haloalkyl, C₁-C₈ haloalkoxy, —CN, —NO₂, —NR⁵R⁶, —C(O)R⁴, —CO₂R⁴, —C(O)NR⁵R⁶, —NR⁵C(O)R⁴, —(CH₂)ₘaryl, —(CH₂)ₘheteroaryl, or —(CH₂)ₘheterocycloalkyl;

R⁴, R⁵, and R⁶ are the same or different and each is H or C₁-C₈ alkyl; and

m and n are the same or different and each is 0 or an integer from 1-5; or a pharmaceutically acceptable salt thereof.

Other compounds described in WO 2017/053769 that may be used as P2Y14 antagonists are represented by the formula (XII):

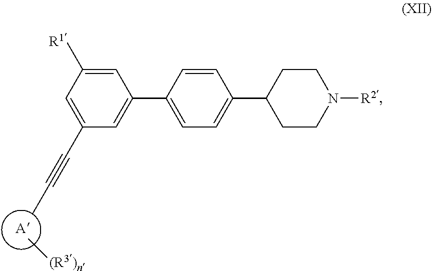

wherein

ring A′ is aryl, heteroaryl, or cycloalkyl;

R^{1′} is —CO₂H, —CO₂(C₁-C₈ alkyl), or a bioisostere of carboxylate;

R^{2′} is H, C₁-C₈ alkyl, C₂-C₈ alkenyl, C₂-C₈ alkynyl, C₃-C₆ cycloalkyl, C₃-C₆ cycloalkylalkyl, hydroxyalkyl, C₁-C₈ haloalkyl, cyanoalkyl, aryl, heteroaryl, heterocycloalkyl, —(CH₂)ₘ′aryl, —(CH₂)ₘ′heteroaryl, or —(CH₂)ₘ′heterocycloalkyl;

each R^{3′} is the same or different and each is C₁-C₈ alkyl, C₂-C₈ alkenyl, C₃-C₆ cycloalkyl, hydroxy, hydroxyalkyl, C₁-C₈ alkoxy, C₃-C₆ cycloalkyloxy, aryloxy, halo, C₁-C₈ haloalkyl, C₁-C₈ haloalkoxy, —CN, —NO₂, —NR^{5′}R^{6′}, —C(O)R^{4′}, —CO₂R^{4′}, —C(O)NR^{5′}R^{6′}, —NR^{5′}C(O)R^{4′}, —(CH₂)_{m′}aryl, —(CH₂)_{m′}heteroaryl, or —(CH₂)_{m′}heterocycloalkyl;

R^{4′}, R^{5′}, and R^{6′} are the same or different and each is H or C₁-C₈ alkyl; and

m′ and n′ are the same or different and each is 0 or an integer from 1-5; or a pharmaceutically acceptable salt thereof.

The P2Y14 antagonist may be a prodrug, analog, derivative, or pharmaceutically acceptable salt of PPTN or of any other active compound that inhibits P2Y14.

Any of the compounds described above may be provided as a pharmaceutically acceptable salt. For example and without limitation, the pharmaceutically acceptable salt may include one or more of 2-hydroxy-ethanesulfonate, 2-naphthalenesulfonate, 3-phenylpropionate, acetate, adipate, alginate, andvalerate, ascorbate, aspartate, benzenesulfonate, benzoate, bicarbonate, bisulfate, bitartrate, borate, bromide, butyrate, camphorate, camphorsulfonate, camsylate, carbonate, chloride, citrate, clavulanate, cyclopentanepropionate, digluconate, dihydrochloride, diphosphate, dodecylsulfate, edetate, edisylate, estolate, esylate, ethanesulfonate, formate, fumarate, gluceptate, glucoheptonate, gluconate, glutamate, glycerophosphate, glycollylarsanilate, hemisulfate, heptanoate, hexanoate, hexylresorcinate, hydrabamine, hydrobromide, hydrochloride, hydroiodide, hydroxynaphthoate, iodide, isothionate, lactate, lactobionate, laurate, laurylsulfate, malate, maleate, malonate, mandelate, mesylate, methanesulfonate, methylbromide, methylnitrate, methylsulfate, mucate, napsylate, nicotinate, nitrate, N-methylglucamineammoniumsalt, oleate, oleate, oxalate, oxalate, palmitate, pamoate (embonate), pantothenate, pectinate, persulfate, phosphate, picrate, pivalate, polygalacturonate, propionate, p-toluenesulfonate, salicylate, stearate, subacetate, succinate, sulfate, tamiate, tartrate, teoclate, thiocyanate, tosylate, triethiodide, undecanoate, and valerate. Other pharmaceutically acceptable salts include nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Representative alkali or alkaline earth metal salts include sodium, lithium, potassium, calcium, magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

Agents that Increase Solubility of P2Y14 Antagonists

The formulations of the invention include one or more agents that increase the solubility of a P2Y14 antagonist, such as PPTN, in an aqueous medium. Examples of suitable agents include sulfobutyl ether beta-cyclodextrin (SBECD) or α-tocopherol polyethylene glycol succinate (TPGS).

The presence of the agent may allow the formulation to contain a certain concentration of the P2Y14 antagonist. Thus, the formulation, including the P2Y14 antagonist and the agent, may contain the P2Y14 antagonist at ≥0.001 μg/ml, ≥0.002 μg/ml, ≥0.005 μg/ml, ≥0.01 μg/ml, ≥0.02 μg/ml, ≥0.05 μg/ml, ≥0.1 μg/ml, ≥0.2 μg/ml, ≥0.5 μg/ml, ≥1 μg/ml, ≥2 μg/ml, ≥5 μg/ml, ≥10 μg/ml, ≥20 μg/ml, ≥50 μg/ml, ≥100 μg/ml, ≥200 μg/ml, ≥500 μg/ml, ≥1 mg/ml, ≥2 mg/ml, ≥5 mg/ml, ≥10 mg/ml, from about 1 μg/ml to about 20 mg/ml, from about 2 μg/ml to about 20 mg/ml, from about 5 μg/ml to about 20 mg/ml, from about 10 μg/ml to about 20 mg/ml, from about 20 μg/ml to about 20 mg/ml, from about 50 μg/ml to about 20 mg/ml, from about 100 μg/ml to about 20 mg/ml, from about 200 μg/ml to about 20 mg/ml, from about 500 μg/ml to about 20 mg/ml, from about 1 mg/ml to about 20 mg/ml, from about 2 mg/ml to about 20 mg/ml, from about 5 mg/ml to about 20 mg/ml, from about 1 μg/ml to about 10 mg/ml, from about 2 μg/ml to about 10 mg/ml, from about 5 μg/ml to about 10 mg/ml, from about 10 μg/ml to about 10 mg/ml, from about 20 μg/ml to about 10 mg/ml, from about 50 μg/ml to about 10 mg/ml, from about 100 μg/ml to about 10 mg/ml, from about 200 μg/ml to about 10 mg/ml, from about 500 μg/ml to about 10 mg/ml, from about 1 mg/ml to about 10 mg/ml, from about 2 mg/ml to about 10 mg/ml, from about 5 mg/ml to about 10 mg/ml, from about 1 μg/ml to about 5 mg/ml, from about 2 μg/ml to about 5 mg/ml, from about 5 μg/ml to about 5 mg/ml, from about 10 μg/ml to about 5 mg/ml, from about 20 μg/ml to about 5 mg/ml, from about 50 μg/ml to about 5 mg/ml, from about 100 μg/ml to about 5 mg/ml, from about 200 μg/ml to about 5 mg/ml, from about 500 μg/ml to about 5 mg/ml, from about 1 mg/ml to about 5 mg/ml, from about 2 mg/ml to about 5 mg/ml, from about 1 μg/ml to about 2 mg/ml, from about 2 μg/ml to about 2 mg/ml, from about 5 μg/ml to about 2 mg/ml, from about 10 μg/ml to about 2 mg/ml, from about 20 μg/ml to about 2 mg/ml, from about 50 μg/ml to about 2 mg/ml, from about 100 μg/ml to about 2 mg/ml, from about 200 μg/ml to about 2 mg/ml, from about 500 μg/ml to about 2 mg/ml, or from about 1 mg/ml to about 2 mg/ml.

The agent may promote solubility of the P2Y14 antagonist at a near-neutral pH. Thus, the formulation, including the P2Y14 antagonist and the agent, may have a pH of >4.0, >4.5, >5.0, >5.5, >6.0, >6.5, >7.0, >7.5, >8.0, from about 4.0 to about 9.0, from about 5.0 to about 9.0, from about 6.0 to about 9.0, from about 7.0 to about 9.0, from about 4.0 to about 8.0, from about 5.0 to about 8.0, from about 6.0 to about 8.0, from about 7.0 to about 8.0, from about 4.0 to about 7.0, from about 5.0 to about 7.0, from about 6.0 to about 7.0, about 5.0, about 5.5, about 6.0, about 6.5, about 7.0, about 7.5, or about 8.0.

The agent may be present in the formulation at a certain concentration. For example, the agent may be present in the formulation at less than about 40%, less than about 35%, less than about 30%, less than about 25%, less than about 20%, less than about 15%, less than about 10%, less than about 5%, less than about 2%, less than about 1%, less than about 0.5%, less than about 0.2%, less than about 0.1%, less than about 0.05%, less than about 0.02%, less than about 0.01%, less than about 0.005%, less than about 0.002%, less than about 0.001%, from about 0.001% to about 0.01%, from about 0.003% to about 0.03%, from about 0.01% to about 0.1%, from about 0.03% to about 0.3%, from about 0.1% to about 1%, from about 0.3% to about 3%, from about 1% to about 10%, from about 2% to about 10%, from about 3% to about 10%, from about 5% to about 10%, from about 5% to about 12%, from about 5% to about 15%, from about 5% to about 20%, from about 7.5% to about 10%, from about 7.5% to about 12%, from about 7.5% to about 15%, from about 7.5% to about 20%, from about 10% to about 12%, from about 10% to about 15%, or from about 10% to about 20%.

The agent may improve the stability of the P2Y14 antagonist. For example, the agent may increase the half-life of the P2Y14 antagonist by about 10%, about 25%, about 50%, about 100%, about 200%, about 500%, about 1000%, or more.

Other Properties of Formulations

The formulations of the inventions may contain additional ingredients.

The formulation may contain salts and/or buffering agents. The buffering agent may be phosphate. The salt may be sodium chloride or potassium chloride. The formulation may contain saline or phosphate-buffered saline. Other salts and buffering agents are described in more detail below.

The formulation may contain dimethyl sulfoxide (DMSO). The formulation may contain DMSO at or below a certain concentration. For example, DMSO may be present in the formulation at less than about 10%, less than about 5%, less than about 3%, less than about 2%, less than about 1%, less than about 0.5%, less than about 0.3%, less than about 0.2%, or less than about 0.1%.

The formulation may be substantially free of solvents or other chemicals that are not suitable for administration to a subject. For example, the formulation may be substantially free of dimethylacetamide (DMAc), ethanol, N-methylpyrrolidone (NMP), and/or polyethylene glycol (PEG).

The formulation may be a pharmaceutical composition. A pharmaceutical composition may be in a form suitable for oral use, for example, as tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions, and such compositions may contain one or more agents selected from sweetening agents, flavoring agents, coloring agents and preserving agents, in order to provide pharmaceutically elegant and palatable preparations. Tablets contain the compounds in admixture with non-toxic pharmaceutically acceptable excipients which are suitable for the manufacture of tablets. These excipients may be for example, inert diluents, such as lactose; granulating and disintegrating agents, for example corn starch, or alginic acid; binding agents, for example starch, gelatin or acacia, and lubricating agents, for example magnesium stearate, stearic acid or talc.

The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release. Preparation and administration of compounds is discussed in U.S. Pat. No. 6,214,841 and U.S. Pub. 2003/0232877, which are incorporated by reference herein in their entirety.

Formulations for oral use may also be presented as hard gelatin capsules in which the compounds are mixed with an inert solid diluent, such as kaolin. The formulations may be presented as soft gelatin capsules in which the compounds are mixed with water or an oil medium, for example peanut oil, liquid paraffin or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol, or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

The pharmaceutical composition may be formulated for intravenous injection or infusion or subcutaneous administration. For example, the compositions may be dissolved, suspended or emulsified. The compositions may also be lyophilized, and the lyophilized material may be used to prepare a formulation for injection. Suitable solvents for injectable formulations include, for example and without limitation, water, physiological saline solution, alcohols, e.g. ethanol, propanol, glycerol, sugar solutions, such as hexose or mannitol solutions, and mixtures of the aforementioned solvents. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic monoglycerides or diglycerides, and fatty acids, including oleic acid.

The pharmaceutical composition may be formulated for delivery of a compound that is insoluble or poorly soluble in water. Examples of such formulations include nanoparticles, microparticles, nanosuspensions, phospholipid-coated microcrystals, emulsions, and stable aqueous formulations. Formulations for delivery of insoluble or poorly soluble compounds are known in the art and described in, for example, U.S. Pat. Nos. 5,091,187; 5,858,410; 8,313,777; 9,308,180; U.S. Publication No. 2002/0012704; U.S. Publication No. 2003/0027858; U.S. Publication No. 2008/0166411; U.S. Publication No. 2010/0093872; U.S. Publication No. 2013/0115165; International Publication No. WO 2014/165660; Pace S. et al., “Novel injectable formulations of insoluble drugs”, Pharm. Tech, 1999, 23:116-134; and Panagiotou T. et al., “Production of stable nanosuspensions using microfluidics reaction technology”, Nanotech. 2007, 4:246-249, ISBN 1420063766, the contents of each of which are incorporated herein by reference.

Pharmaceutical compositions may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol. sorbitol, mannitol and polyethylene glycol; esters, such asethyl oleate and ethyllaurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

### Methods of Treating Renal Disorders

Renal Disorders and Disorders Associated with Renal Inflammation

The invention provides methods of treating kidney disorders or conditions associated with renal inflammation by providing to a subject a formulation of the invention. The methods are useful for treating any disease, disorder, or condition that is associated with renal inflammation or for which treating or preventing renal inflammation provides a therapeutic benefit. The use of P2Y14 antagonists to treat kidney disorders or conditions associated with renal inflammation is known in the art and described in, for example, U.S. Pat. No. 9,891,236; International Patent Publication No. WO 2017/165665; and co-owned, co-pending U.S. Application Nos. 62/658,241; 62/659,438; 62/662,932; 62/664,464; 62/675,256; 62/682,270, the contents of each of which are incorporate herein. Thus, the formulations of the invention may be used to treat any disease, disorder, or condition described in the foregoing patent applications.

For example and without limitation, the disease, disorder, or condition may be or include accumulation of mesangial matrix, acute interstitial nephritis, acute kidney injury (AKI), including AKI associated with cardiac surgery, acute tubular necrosis (ATN), Alport syndrome, atherosclerosis, atherosclerotic renal artery stenosis, an autoimmune disorder, such us systemic lupus erythematosus, autosomal dominant polycystic kidney disease (ADPKD), benign prostatic hyperplasia, bladder stones, cancer, including cancer of the bladder, ureters, or prostate, cardiac surgery, cell apoptosis, chronic kidney disease, chronic tubulointerstitial nephritis, delayed graft function (DGF), including DGF-renal, diabetes, diabetic nephropathy, including type 1 diabetic nephropathy (T1D nephropathy), end-stage renal disease, Focal segmental glomerulosclerosis (FSGS), glomerular basement membrane thickening, glomerular hyperfiltration, glomerular and tubular epithelial hypertrophy, glomerulonephritis, glomerulosclerosis, heart failure, hemolytic-uremic syndrome, hypertension, IgA nephropathy (also called Berger's disease), infection, injury, ischemia, ischemia/reperfusion injury, ischemic nephropathy, kidney hypoxia, kidney stones, kidney transplantation, liver cirrhosis, methyl melonic acidosis (MMA), microalbuminuria, obstructed urinary catheter, proteinuria, reduced creatinine clearance, reduced glomerular filtration rate, reflux nephropathy, renal vein thrombosis, rhabdomyolysis, tumor lysis syndrome, vascular occlusion, or vasculitis.

The methods may treat or prevent renal inflammation associated with acute kidney injury (AKI). AKI may be assessed by any suitable standard. Several standards for acute kidney injury are known in the art, such as the criteria provided by the Acute Kidney Injury Network (AKIN); Kidney Disease Improving Global Outcomes (KDIGO); and Risk, Injury, Failure, Loss, and End-stage Kidney (RIFLE). AKI may be categorized or staged according to the AKI, KDIGO, or RIFLE criteria. For example, a subject may be deemed to have stage 1, stage 2, or stage 3 AKI, or a subject may be deemed to have risk, injury, failure, or loss. The standard may apply to an adult, pediatric, newborn, neonatal, infant, child, adolescent, pre-teen, teenage, or elderly subject.

Standards typically include measurements of serum creatinine (SCr) concentrations, urine output, or glomerular filtration rate (GFR). Standards may include multiple parameters, e.g., combinations of the aforementioned standards. A subject may be deemed to have AKI, or a stage or category thereof, when she has abnormally high SCr concentration, abnormally low urine output, abnormally low GFR, or any combination thereof. Standards may be absolute, e.g., they may require a value above or below a defined threshold value. Alternatively, standards may be relative, e.g., they may require an increase or decrease relative to a baseline value. Standards for different parameters, e.g., abnormally high SCr concentration abnormally low urine output, or abnormally low GFR, may independently be absolute or relative.

Standards for acute kidney injury may include a temporal component. For example, a subject may be deemed to have AKI when an elevated SCr concentration is measured at some interval following a preceding event. The preceding event may be cardiac surgery, cardiac arrest, admission to a hospital, clinic, medical facility, or any unit thereof. The interval may be 1 hour, 2 hours, 4 hours, 6 hours, 8 hours, 12 hours, 24 hours, 36 hours, 48 hours, or 72 hours. A subject may be deemed to have AKI when urine output is measured across some interval, such as 1 hour, 2 hours, 4 hours, 6 hours, 8 hours, 12 hours, 24 hours, 36 hours, 48 hours, or 72 hours.

For example and without limitation, a standard for reduced urine output associated with AKI may be less than 0.5 mLUkg/h for 6-12 hours, less than 0.5 mLUkg/h for at least 12 hours, or less than 0.3 mLUkg/h for 24 hours, or anuria for at least 12 hours.

For example and without limitation, a standard for elevated SCr concentration associated with AKI may be a SCr concentration of at least 0.3 mg/dl, a SCr concentration of at least 1 mg/dl, a SCr concentration of at least 4 mg/dl, a SCr concentration of at least 26.5 μmol/l, or a SCr concentration of at least 353.6 μmol/l. For example and without limitation, a standard for elevated SCr concentration associated with AKI may be an increase of 50% over baseline, an increase of 100% over baseline, or an increase of 200% over baseline.

For example and without limitation, a standard for GFR associated with AKI may be a GFR of less than 35 ml/min per 1.73 mm2. For example and without limitation, a standard for GFR associated with AKI may be a decrease of at least at least 25% relative to a baseline, a decrease of at least at least 50% relative to a baseline, or a decrease of at least at least 75% relative to a baseline.

The methods may treat or prevent renal inflammation associated with reperfusion injury. Reperfusion injury, which is also called reperfusion insult, ischemia-reperfusion injury, and reoxygenation injury, is the tissue damage that results when blood supply to the tissue is restored after a period of ischemia or lack of oxygen. The sudden influx of nutrients and oxygen after a bout of ischemia, anoxia, or hypoxia produces a high level of reactive oxygen species that exceeds the tissue's detoxification capacity. The oxidative stress is associated with microvascular injury due to increased permeability of capillaries and arterioles that allows fluid to penetrate the tissue more readily. In addition, white blood cells in the returning blood respond to damaged tissue by releasing inflammatory factors.

Reperfusion injury can occur following any surgery that limits blood supply to an organ. In particular, reperfusion injury is a risk following cardiac procedures due to changes in blood during the procedure. Reperfusion injury is also a major concern in organ transplantation procedures due to the lack of blood flow to the organ while it is being transported.

Certain organs are particularly vulnerable to reperfusion injury. For example, reperfusion injury contributes to the brain's ischemic cascade in stroke or brain trauma, and also plays a role in brain damage following cardiac arrest. The heart, kidneys, lungs, and liver may also be affected by reperfusion injury.

Without wishing to be bound by a particular theory, P2Y14 antagonists may prevent or minimize reperfusion injury by preserving microvascular integrity. Therefore, it may be advantageous to provide a P2Y14 antagonist, such as any of the P2Y14 antagonists described above, to a patient before the patient undergoes a surgery that is likely to cause reperfusion injury. The surgery may be a cardiac procedure, such as any of the cardiac procedures described above. The P2Y14 antagonist may be provided by any mechanism described above.

Methods of Providing Formulations

Providing the formulation to the subject may include administering it to the subject. The formulation may be administered by any suitable means. For example and without limitation, the formulation may be administered buccally, by injection or infusion, dermally, enterally, enterally, intraarterially, intravenously, nasally, orally, parenterally, pulmonarily, rectally, subcutaneously, topically, transdermally, vaginally, or with or on an implantable medical device (e.g. stent or drug-eluting stent or balloon equivalents). The formulation may be provided directly to the kidney via in vitro perfusion of the renal artery.

The P2Y14 antagonist may be provided at any suitable dosage. For example and without limitation, the P2Y14 antagonist may be provided at from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, or from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, or from 4.5 g/kg body weight to 5 g/kg body weight. These doses may be administered at a single time or multiple times each day, or may be administered continuously, for example by continuous intravenous infusion. For example and without limitation, the P2Y14 antagonist may be provided at from 0.001 mg/kg body weight/day (mg/kg/day) to 5 g/kg body weight/day. In some embodiments, the dosage range is from 0.001 mg/kg body weight/day to 1 g/kg body weight/day, from 0.001 mg/kg body weight/day to 0.5 g/kg body weight/day, from 0.001 mg/kg body weight/day to 0.1 g/kg body weight/day, from 0.001 mg/kg body weight/day to 50 mg/kg body weight/day, from 0.001 mg/kg body weight/day to 25 mg/kg body weight/day, from 0.001 mg/kg body weight/day to 10 mg/kg body weight/day, from 0.001 mg/kg body weight/day to 5 mg/kg body weight/day, from 0.001 mg/kg body weight/day to 1 mg/kg body weight/day, from 0.001 mg/kg body weight/day to 0.1 mg/kg body weight/day, or from 0.001 mg/kg body weight/day to 0.005 mg/kg body weight/day. Alternatively, in some embodiments the dosage range is from 0.1 g/kg body weight/day to 5 g/kg body weight/day, from 0.5 g/kg body weight/day to 5 g/kg body weight/day, from 1 g/kg body weight/day to 5 g/kg body weight/day, from 1.5 g/kg body weight/day to 5 g/kg body weight/day, from 2 g/kg body weight/day to 5 g/kg body weight/day, from 2.5 g/kg body weight/day to 5 g/kg body weight/day, from 3 g/kg body weight/day to 5 g/kg body weight/day, from 3.5 g/kg body weight/day to 5 g/kg body weight/day, from 4 g/kg body weight/day to 5 g/kg body weight/day, or from 4.5 g/kg body weight/day to 5 g/kg body weight/day. Effective doses may be estimated from dose-response relationships derived from in vitro or animal model test bioassays or systems or from clinical trials of the P2Y14 antagonist. The dosage should not be so large as to cause unacceptable adverse side effects.

Improvement of Renal Function

Providing formulations containing a P2Y14 antagonist may improve renal function. For example, renal function may be improved by at least 10%, at least 20%, at least 30%, at least 40%, at least 50%, at least 60%, at least 70%, at least 80%, at least 90%, at least 100%, at least 200%, or at least 300%. Measurable markers of renal function, are well known in the medical and veterinary literature and to those of skill in the art, and include, but are not limited to, blood urea nitrogen or “BUN” levels (both static measurements and measurements of rates of increase or decrease in BUN levels), serum creatinine levels (both static measurements and measurements of rates of increase or decrease in serum creatinine levels), measurements of the BUN/creatinine ratio (static measurements of measurements of the rate of change of the BUN/creatinine ratio), urine/plasma ratios for creatinine, urine/plasma ratios for urea, glomerular filtration rates (GFR), serum concentrations of sodium (Na+), urine osmolarity, daily urine output, albuminuria, proteinuria, and the like. Of the above, measurements of the plasma concentrations of creatinine and/or urea or BUN are particularly important and useful readouts of renal function.

### EXAMPLES

### Example 1

Solutions containing PPTN were analyzed to determine the limit of quantitation by HPLC. Throughout the examples, PPTN may be referred to as KB-1801; the two terms are interchangeable.

To analyze HPLC mobility, A 0.1% trifluoroacetic acid solution was used as mobile phase A, and acetonitrile was used as mobile phase B.

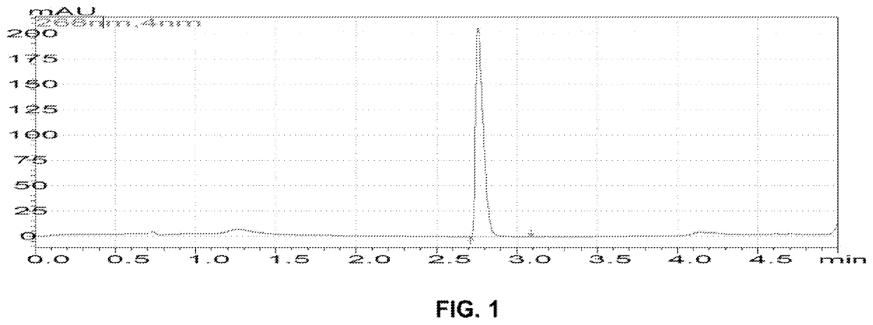

FIG. 1 is a graph showing HPLC analysis of PPTN. 1 μL of a 0.1 mg/mL solution of PPTN in 50/50 acetonitrile/water was injected into the column, and the flow-through was measured by UV absorption at 268 nm. Retention time of PPTN was 2.759 minutes, and the peak area was 679,876.

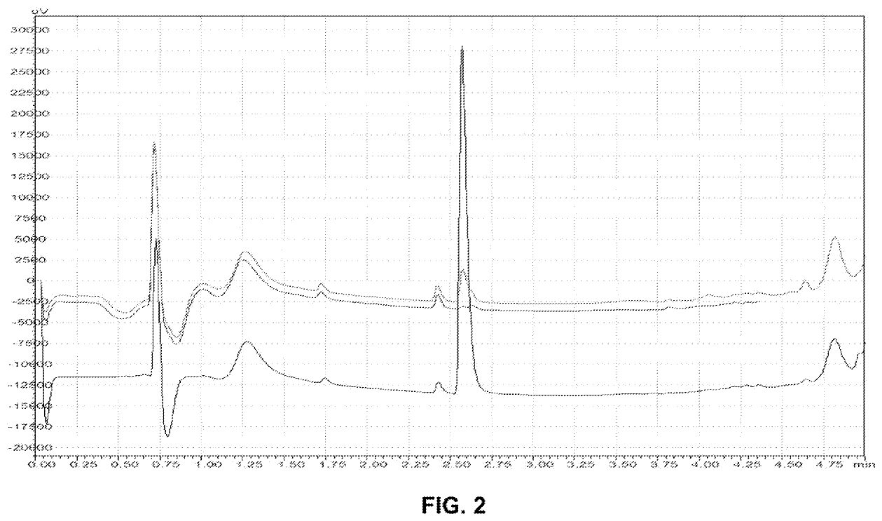

FIG. 2 is a graph showing HPLC analysis of PPTN. 40 μL of a 0.01 mg/mL solution of PPTN in 50/50 acetonitrile/water injected into the column, and the flow-through was measured by UV absorption at 268 nm. Blue trace, blank sample; purple trace, 200 ng/mL; black trace, 2000 ng/mL. Results are summarized in Table 1.

TABLE 1

| [KB-1801] | Peak area |
|---|---|
| Blank | <1000 |
| 200 ng/mL | 14,480 |
| 2000 ng/mL | 136,353 |

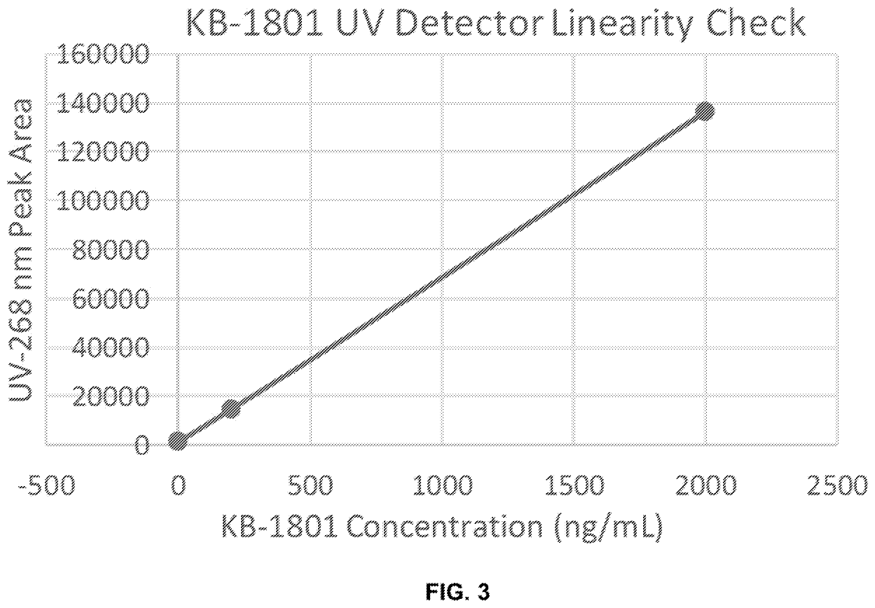

FIG. 3 is a graph of UV absorption at 268 nm vs. concentration of PPTN (KB-1801).

### Example 2

The solubility of PPTN in various aqueous and organic solvents was tested. Test media were prepared, and 5 mg/mL solid PPTN (HCl salt) was added at room temperature. If all PPTN dissolved under these conditions, additional PPTN was added to a total of 10 mg/mL. After 24 hours, undissolved material was removed, and the concentration of PPTN in the supernatant was analyzed by HPLC. Results are summarized in Table 2.

TABLE 2

| | | | Solubility | |
|---|---|---|---|---|
| Solvent Name | Solvent Volume | Observations | Estimate | Remarks |
| water w/ 1% | 50 mL, then 2x and | Cloudy at 2XD | 0.1-0.5 mg/mL | |
| DMAc | 10x dilutions | Clear at 10XD | (pH 2-3) | |
| DMAc | 0.5 mL | Clear solution | >100 mg/mL | |
| | | | (pH 1-2) | |
| NMP | 0.5 mL | Clear solution | >100 mg/mL | |
| | | | (pH 1-2) | |
| PEG 400 | 1.0 mL | Cloudy and viscous | ~25 mg/mL | Clear solution |
| | | solution | (pH ~2) | achieved with |
| | | | | another 1.0 mL |
| ethanol | 2.0 mL | Clear solution after | ~25-30 mg/mL | |
| | | sonication | (pH ~2) | |
| propylene | 1.0 mL | Slightly and viscous | 40-50 mg/mL | Clear solution |
| glycol | | solution | (pH ~2) | achieved with a |
| | | | | few more drops |

DMAc = dimethylacetamide; NMP = N-methylpyrrolidone; PEG = polyethyleneglycol

The most effective solvents were dimethylacetamide and N-methylpyrrolidone>However, the safety and utility of these solvents for formulations for intravenous infusion are unknown.

The solubility of PPTN at pH ranges suitable for intravenous injection was analyzed. PPTN (HCl salt) was dissolved in DMSO to make a stock solution, and the stock solution was diluted into various aqueous solutions. The pH values of the diluted aqueous solutions were measured, and the solutions were observed for indications of insolubility. The solutions were then adjusted to achieve a pH ≥5.0, and the solutions were observed again. Results are provided in Table 3 and Table 4.

TABLE 3

| | | pH | Solution | |
|---|---|---|---|---|
| | | (with pH | appearance | |
| [KB-1801] | Solvent Composition | paper) | at 24 hours | % recovery |
| 0.015 | 1.5 mg DMSO | pH 4-5 | Clear | 86% |
| mg/mL | stock solution | | | |
| (~29 μM) | diluted 100x in water | | | |
| 5.0 mg/mL | 1.5 mg DMSO stock | pH 5-6 | Hazy | 0.12% |
| | solution diluted | | | |
| | 100x in PBS (20 mM) | | | |

TABLE 4

| | | pH | |
|---|---|---|---|
| | | (with pH | Remarks when |
| [KB-1801] | Solvent Composition | paper) | pH adjusted to 5-5.5 |
| 1.0 mg/mL | DMAc/Water | pH ~2 | Became cloudy |
| | (10/90, v/v) | | |
| 5.0 mg/mL | DMAc/Water | pH 1-2 | Became very cloudy |
| | (10/90, v/v) | | |
| 5.0 mg/mL | NMP/Water | pH 1-2 | Became very cloudy |
| | (10/90, v/v) | | |
| 5.0 mg/mL | Propylene Glycol/Water | pH 1-2 | Became very cloudy |
| | (10/90, v/v) | | |
| 5.0 mg/mL | DMAc/Water | pH 1-2 | Became slightly cloudy; |
| | (10/90, v/v) with | | became clear |
| | ~0.1% Tween 80 | | after sonication |

α-Tocopherol polyethylene glycol succinate (TPGS) was tested for enhancement of solubility of PPTN at neutral pH. A solution containing 5 mg/mL PPTN and 4% TPGS was made by mixing components as indicated in Table 5 with stirring and heating.

TABLE 5

| | | | Composition in |
|---|---|---|---|
| Compound | | | Conc., Weight or |
| Name | Brand Name/Lot # | Amount | volume % |
| KB-1801 API | WuXi, | 49.6 mg | 5 mg/mL |
| | Batch # ET21484-34-P1 | | (0.01 M or 10 mM) |
| TPGS solution | 20% TPGS in water | 2.0 mL | 4 wt % |
| in D.I. water | | | Resulting clear |
| | | | solution |
| Phosphate buffer | Potassium dibasic | 2.0 mL | 30 vol % |
| | phosphate, | | Resulting clear |
| | 20 mM, pH = 8.3 | | solution |
| Buffered saline | | 6.0 mL | 60% |
| Total Volume | — | — | 10 mL (100 vol %) |
| Solution pH | | | Final pH: 7.0-7.4 |

To create a solution suitable for in vitro studies, the solution containing 5 mg/mL PPTN and 4% TPGS created above was diluted into phosphate-buffered saline by mixing components as indicated in Table 6.

TABLE 6

| | | Composition in Conc., |
|---|---|---|
| Compound Name | Amount | Weight or volume % |
| 5 mg/ml KB-1801 solution | 1.0 mL | 100 μM |
| | | Resulting clear solution |
| Buffered Saline | 99.0 mL | |
| TPGS | 100xD | 0.04 wt % |
| Total Volume | — | 100 mL (100 vol %) |
| Solution pH | | Final pH: 7.4 |

### Example 3

The loss of PPTN due to adherence to surfaces of vessels was analyzed. PPTN is a hydrophobic compound that may adhere to plastics and other surfaces. To test how much PPTN is lost due to surface adhesion, an aqueous solution containing 200 ng/ml PPTN and less than 0.1% acetonitrile was incubated in the absence of solubilizing agent in various vessels for 24 hours. The results are shown in Table 7.

TABLE 7

| Sample | | 1^{st} | 2^{nd} | 3^{rd} | | % recovery | % recovery |
|---|---|---|---|---|---|---|---|
| name | Material type | PA* | PA | PA | Average | vs. standard | vs. control |
| Standard | New glass volumetric flask | 13570 | 13026 | 13790 | 13462 | 100% | N/A |
| Control 48 h | Glass scintillation vial | 3662 | 3252 | | 347 | 26% | 100% |
| 1 | polyetheretherketone tubing | 1004 | 511 | | 758 | 6% | 22% |
| 2 | Corning pipette tip | 855 | 662 | | 759 | 6% | 22% |
| 4 | Eppendorf orange conical tube | 886 | 811 | | 849 | 6% | 25% |
| 5 | Eppendorf clear conical tube | 752 | 569 | | 661 | 5% | 19% |
| 6 | Versilon SE200 Tygon tubing | 260 | 294 | | 277 | 2% | 8% |

*PA: peak area of UV absorption at 273 nm

An aqueous solution containing 5 mg/mL PPTN, 0.1% DMSO, and 0.04% TPGS in phosphate-buffered saline was incubated in the absence of additional solubilizing agent in various vessels for 16-24 hours. Results are shown in Table 8.

TABLE 8

| Sample name | Material type | % recovery |
|---|---|---|
| 1 | Plastic vial | 116% |
| 2 | Glass scintillation vial | 98% |
| 3 | polyetheretherketone tubing | 105% |

The results show that a substantial amount of PPTN is lost from a low-concentration solution that does not contain TPGS but not from high concentration solution that contains TPGS.

### Example 4

The effect of pH on the solubility of PPTN in an aqueous medium was analyzed. Solutions of PPTN in either water or phosphate buffer at various pH values were incubated with agitation for 24-48 hours at room temperature then filtered to remove precipitates, and the PPTN concentration of the filtered solution was analyzed. Results are shown in Table 9.

TABLE 9

| | [Phosphate | Solution | Room | [KB-1801.HCl] |
|---|---|---|---|---|
| pH* | buffer] (mM) | agitation time | Temperature | by HPLC Analysis |
| 1.9 | 10 | 24-48 h | ~22° C. | 0.0008 mg/mL/1.6 μM |
| 2.8 | 10 | 24-48 h | ~22° C. | 0.0255 mg/mL/50 μM |
| 4.8 | 0 (DIW) | >24 h | ~25° C. | ~0.1 mg/mL/195 μM |
| | | | (from initial | |
| | | | sonication | |
| 6.6 | 10 | 24-48 h | ~22° C. | 0.0001 mg/mL/0.2 μM |
| 8.2 | 10 | 24-48 h | ~22° C. | 0.0001 mg/mL/0.2 μM |
| 10.4 | 10 | 24-48 h | ~22° C. | 0.0/0 μM |

### Example 5

Sulfobutylether-β-cyclodextrin (SBECD) was tested for the ability to increase the solubility of PPTN in aqueous media.

A stock solution containing 12% SBECD was prepared as follows: 9 mL of saline was added to 1.21 g of SBECD in a 10 mL flask, and the mixture was sonicated for 5 minutes. Saline was then added to achieve a final volume of 10 mL. The pH of the final solution was about 7.

The solubility of PPTN in solutions containing various concentrations of SBECD was analyzed. Excess PPTN was added to saline solutions containing various concentrations of SBECD, insoluble material was removed by filtration, and the PPTN concentration in the filtered solution was analyzed.

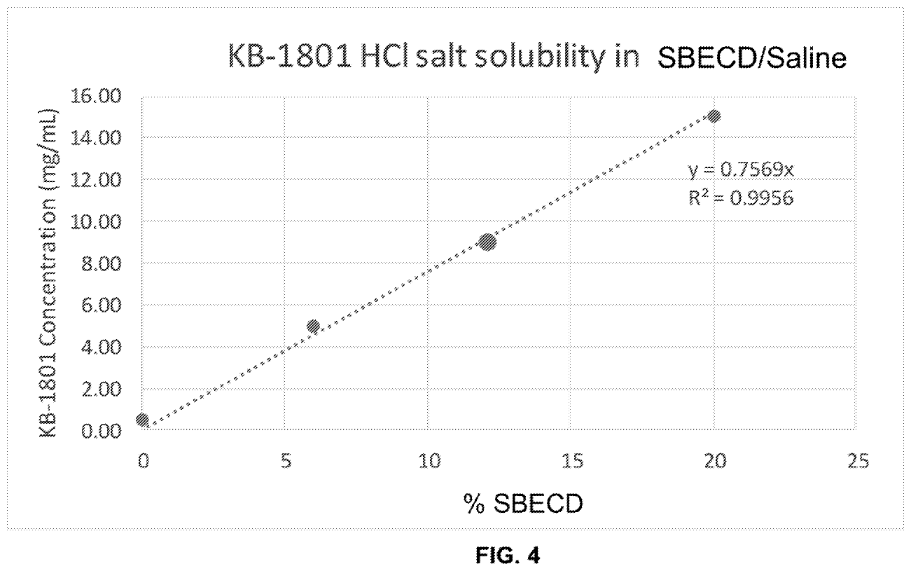

FIG. 4 is graph showing the concentration of PPTN in saturated solutions that contain different amounts of SBECD.

A solution containing 9 mg/mL PPTN was prepared as follows. 4.7 mL of 12% SBECD solution, prepared as described above, was added to a 5 mL flask containing 45 mg PPTN (HCl salt), and the mixture was sonicated with heat for 50 minutes. After sonication, 12% SBECD solution to achieve a final volume of 5 mL. The warm solution appeared clear. The solution was allowed to cool to room temperature and was incubated at room temperature for 28 days. The solution remained clear throughout the entire incubation.

The stability of PPTN in SBECD-containing solutions was analyzed under various storage conditions. 20-mL glass vials containing 10 mL of a solution containing 9 mg/mL PPTN and 12% SBECD, as described above, were incubated under various conditions as described in Table 10.

TABLE 10

| | | Time Points | | | | | |
|---|---|---|---|---|---|---|---|
| Condition | | Initial | 24 h | 7 d | 14 d | 28 d | Objectives/Comments |
| −20° | C. | T0 | X | | | | Freeze/thaw |
| 2-5° | C. | | X | X^{†} | X^{†} | X^{†} | Cold storage/excursion |
| 25° | C. | | X | X | X | X | Ambient storage |
| 40° | C. | | X | X | X | X | ICH Std. accelerated |
| | | | | | | | condition |
| 50° | C. | | X | | | | Exptl. accelerated |
| | | | | | | | condition |

X = time points of pulls/analyses
X^{†} = samples prepared and put on station. HPLC analysis only if solution and/or chemical stability not good at 25° C. time points. Appearance recorded at all time points.

Samples were analyzed by UHPLC-UV as described in Table 11.

TABLE 11

| UPLC | | |
|---|---|---|
| parameters | UPLC parameters | |
| Instrument | Shimadzu Nexera-I LC-2040C-3D (EB-001) | |
| Column | Agilent XDB-C18, 1.8 μm, 4.5 × 50 mm, at 40° C. | |
| UV-PDA | Photo diode array (200-400 nm) | |
| | wv: 220 nm; or alternative wv at 268 to 271 nm | |
| Flow rate | 0.9 mg/mL (pressure: 180 bars) | |
| Injection Volume | 2 μL (for normal concentration at 1.0 mg/mL) | |
| | 40 μL (for low concentrations at ng/mL) | |
| Mobile Phase | A: 0.1% TFA in water | |
| | B: HPLC grade acetonitrile | |
| | Time (min) | B% |
| Gradient | 0 | 10% |
| | 3.0 | 90% |
| | 4.0 | 90% |
| | 4.1 | 10% |
| | 5.1 | 10% |

| | |
|---|---|
| Standard | Dissolved 50.00 mg of KB-1801 HCl salt in 50-mL volumetric flask |
| Preparation | Diluent: Acetonitrile/water 80/20 (v/v) |
| Sample | Pipet 1.0 mL KB-1801 formulation into a 20-mL volumetric flask and |
| Preparation | dilute to volume with HPLC grade water |

Results of stability analysis are shown in Table 12.

TABLE 12

| | [PPTN] | | | | |
|---|---|---|---|---|---|
| Conditions | 0d | 1d | 7d | 14d | 28d |
| −20° C. | | 9.0* | | | |
| 2-5° C. | | 8.74 | 8.69 | 8.98 | 8.65 |
| 25° C. | 8.72 | 8.75 | 8.82 | 8.69 | 8.71 |
| 40° C. | | 8.71 | 8.71 | 9.25** | 8.73 |
| 50° C. | | 8.78 | | | |

*Solution viscosity increased, causing assay variability
**Assay variability (+6%) was observed.

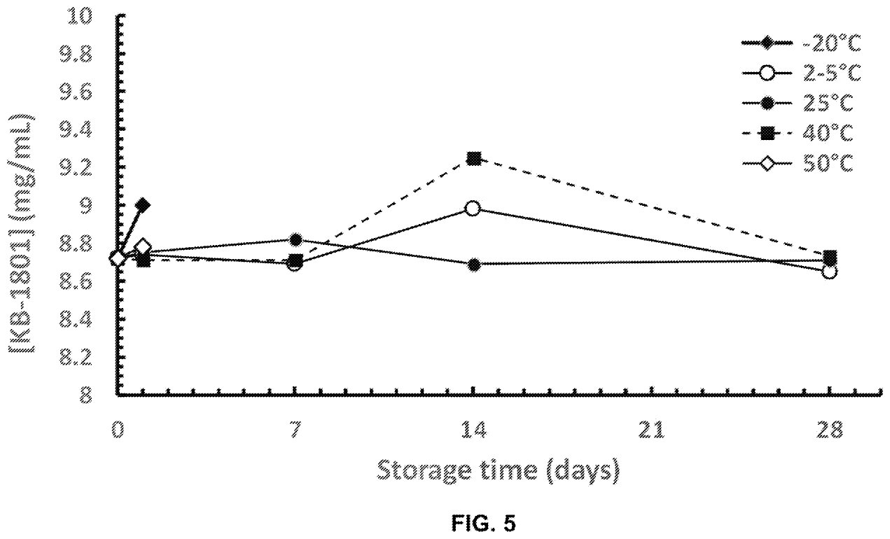

FIG. 5 is a graph of PPTN (KB-1801) concentrations of samples tested in stability assay.

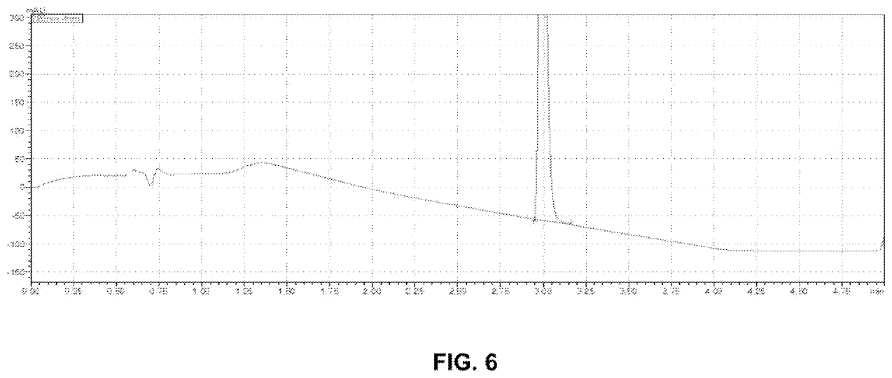

FIG. 6 is a representative UHPLC-UV trace for the sample at 40° C., time=28 days. The peak is PPTN (KB-1801). No degradants were evident.

All solutions retained a clear appearance with no detectable precipitation or turbidity. Freeze/thaw cycles results in an apparent change in viscosity.

The stability of PPTN in SBECD-containing solutions was analyzed at various concentrations of PPTN. A solution containing 9 mg/mL PPTN and 12% SBECD, as described above, was diluted into saline as described in Table 13.

TABLE 13

| Dilution | | |
|---|---|---|
| Factor | Procedure | Result |
| 3x | 1 mL stock mixed with 2 mL saline | Clear solution |
| 10x | 1 mL stock into a 10 mL volumetric | Clear solution |
| | flask and QS with saline | |
| 50x | 1 mL stock into a 50 mL volumetric | Clear solution |
| | flask and QS with saline | |

The stability of high-concentration PPTN solutions containing SBECD were analyzed under conditions used for product manufacturing and distribution. A stock solution containing 8.6 mg/mL PPTN, 12% SBECD in saline was prepared as described above and shipped under refrigerated conditions. Seven days after manufacture, the stock solution was diluted to make dosing solutions for administration as a continuous intravenous infusion as indicated in Table 14 and Table 15.

TABLE 14

| | | KB-1801 | | | | Minimum | Actual | Required |
|---|---|---|---|---|---|---|---|---|
| | | free base | Dose | [KB- | Body | Volume | Batch | Test |
| | # of | Dose Level | Volume | 1801] | Weight | Required | Size | Article |
| Group | Animals | (mg/kg/day) | (mL/kg/day) | (mg/mL) | (kg) | (mL) | (mL) | (mL/prep) |
| Vehicle | 18 | 0.0 | 40.0 | 0.00 | 0.4 | 360.0 | 400 | 0.0 |
| Low | 18 | 10.0 | 40.0 | 0.25 | 0.4 | 360.0 | 400 | 12.0 |
| Mid | 18 | 30.0 | 40.0 | 0.75 | 0.4 | 360.0 | 400 | 35.9 |
| High | 18 | 100.0 | 40.0 | 2.50 | 0.4 | 360.0 | 400 | 119.6 |

TABLE 15

| | KB-1801 | SBECD | | |
|---|---|---|---|---|
| | stock | (12%)/saline | Saline | Total |
| | solution | solution diluent | diluent | volume |
| Group | (mL) | (mL) | (mL) | (mL) |
| Vehicle | 0 | 125.2 | 274.8 | 400.0 |
| Low (10 mg/kg/day) | 12.5 | 112.7 | 274.8 | 400.0 |
| Mid (30 mg/kg/day) | 37.6 | 87.6 | 274.8 | 400.0 |
| High (100 mg/kg/day) | 125.2 | 0 | 274.8 | 400.0 |

Samples of diluted dosing solutions were shipped at ambient temperature and analyzed for PPTN (KB-1801) potency and purity. Results are provided in Table 16.

TABLE 16

| | | | | | Impurity |
|---|---|---|---|---|---|
| | | | Targeted | | profile |
| | | Vial | [KB-1801] | [KB-1801]* | by HPLC |
| # | Sample ID | Label | mg/mL | (mg/mL) | area % |
| 1 | L5224579-0001 | #1 | 0 | 0 | No imp > 0.10 a % |
| 2 | L5224579-0003 | #5 | 0.25 | 0.26 (+4.0%) | No imp > 0.10 a % |
| 3 | L5224579-0005 | #9 | 0.75 | 0.76 (+1.3%) | No imp > 0.10 a % |
| 4 | L5224579-0007 | #13 | 2.5 | 2.56 (+2.4%) | No imp > 0.10 a % |
| 5 | L5224579-0009 | #3 | 0.25 | 0.27 (+8.0%) | No imp > 0.10 a % |
| 6 | L5224579-0011 | #7 | 0.25 | 0.27 (+8.0%) | No imp > 0.10 a % |
| 7 | L5224579-0014 | #11 | 2.5 | 2.56 (+2.4%) | No imp > 0.10 a % |
| 8 | L5224579-0015 | #15 | 2.5 | 2.57 (+2.8%) | No imp > 0.10 a % |

*[KB-1801] determined by HPLC assay, single preparations, duplicate injections.

Assay results show that all samples contained PPTN (KB-1801) at slightly higher (1.6-8.0%) than target concentrations. Samples taken from the top, middle, and bottom of the flasks in which the dilutions were performed had very similar PPTN (KB-1801) concentrations, indicating that the stock formulation is readily diluted into homogeneous solution. No evidence of degradation products was observed in any sample

The osmolarity of a stock solution containing 8.6 mg/mL PPTN, 12% SBECD in saline was analyzed. Results are provided in Table 17.

TABLE 17

| | Measurements (mOsm) | | | | |
|---|---|---|---|---|---|
| | 1 | 2 | 3 | Average | Calculated |
| Saline | 288 | 295 | 288 | 290 | 308 |
| KB-1801 (8.6 mg/mL)/SBECD | 635 | 638 | 637 | 637 | 729 |
| 12%/Saline | | | | | |
| Vehicle SBECD 12%/Saline | 598 | 599 | 600 | 599 | 696 |
| KB-1801 (8.6 mg/mL, as HCl salt) | | | | | 34 |

The pharmacokinetics of PPTN (KB-1801) in male rats receiving single bolus intravenous injections of SBECD-containing solutions were analyzed. Three cohorts of four rats each were given a single dose of PPTN (KB-1801) at 0.85 mg/kg, 2.9 mg/kg, or 8.5 mg/kg in 1 mL dosing volume.

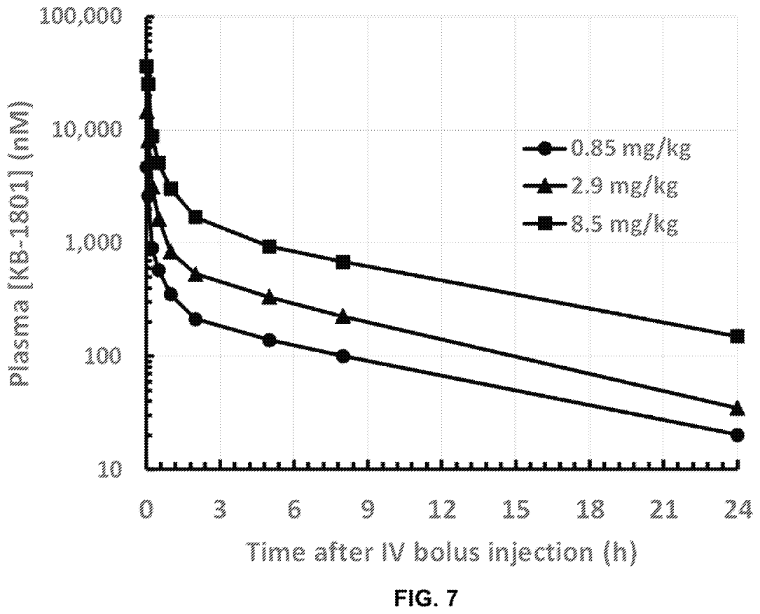

FIG. 7 is a graph of plasma PPTN concentration over time in male rats after a single bolus injection. Dosing amounts are as indicated.

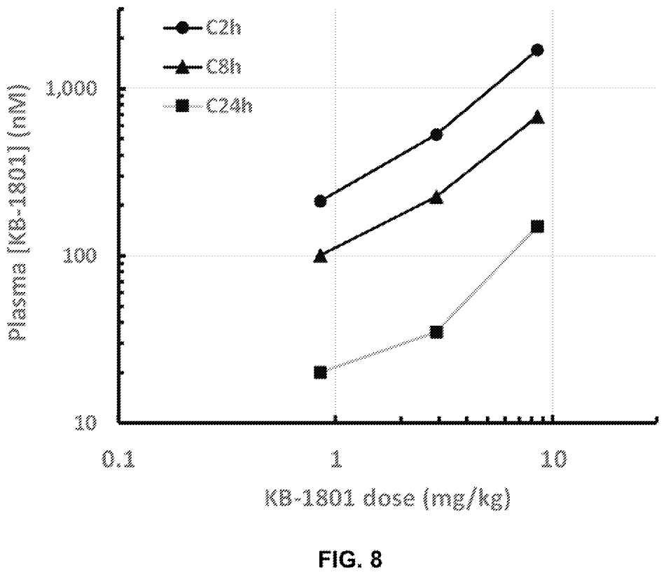

FIG. 8 is a graph of plasma PPTN concentration as a function of dose in male rats after a single bolus injection. Dosing amounts are as indicated.

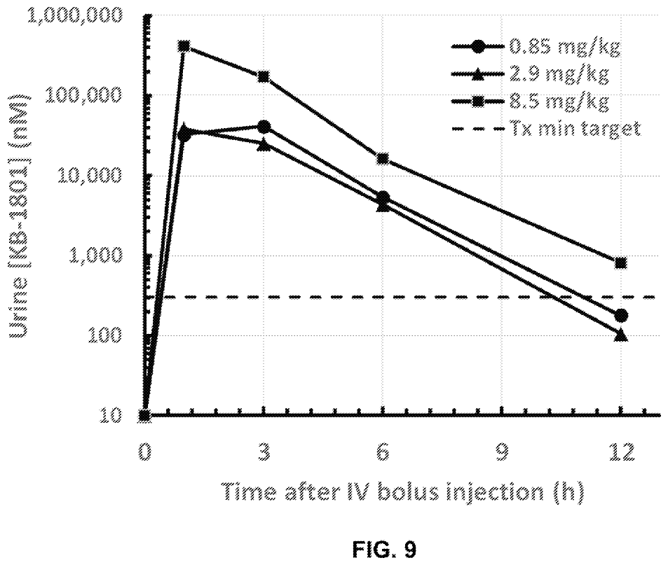

FIG. 9 is a graph of urine PPTN concentration over time in male rats after a single bolus injection. Dosing amounts are as indicated.

The results show that the pharmacokinetics of plasma PPTN were well-described by a 2-compartment model (distribution and elimination), and all PK parameters were linearly dose-related. PPTN was excreted in the urine at concentrations in excess of plasma concentrations and well above those known to inhibit P2Y14 receptor activation in vitro. PPTN/SBECD formulations were well tolerated by all rats, and no in-life adverse events reported.

### Example 6

The safety and pharmacokinetics of PPTN (KB-1801) in rats receiving continuous intravenous injections of SBECD-containing solutions were analyzed. Three cohorts of six male and six female rats each were given continuous intravenous infusion of PPTN (KB-1801) at 10 mg/kg/day, 30 mg/kg/day or 100 mg/kg/day in a dosing solution containing PPTN at 0.25 mg/mL, 0.75 mg/mL or 2.5 mg/mL and SBECD at about 3.8%. Dosing volume was 40 mIkg/day.

KB-1801 was not detectable in pooled plasma of animals infused with vehicle alone. The limit of detection was 5 ng/mL.

Plasma PPTN Concentrations: Results

Plasma concentrations of KB-1801 following a 24-hour intravenous infusion at 10 mg/kg to male Sprague-Dawley rats are shown in Table 18.

TABLE 18

| | | | | | | | Mean | SD | |
|---|---|---|---|---|---|---|---|---|---|
| Time | Animal ID | | | | | | (ng/ | (ng/ | % |
| (h) | 6001 | 6002 | 6003 | 6004 | 6005 | 6006 | mL) | mL) | CV |
| 1 | NQ | NQ | 216.3 | — | — | — | 216.3 | — | — |
| 2 | — | — | — | 327.1 | 232.9 | 267.6 | 275.9 | 47.7 | 17.3 |
| 4 | 368.8 | 227.7 | 537.2 | — | — | — | 377.9 | 155.0 | 41.0 |
| 6 | — | — | — | 507.8 | 319.8 | 422.8 | 416.8 | 94.2 | 22.6 |
| 8 | 429.7 | 355.4 | 464.0 | — | — | — | 416.4 | 55.5 | 13.3 |
| 24 | — | — | — | 931.8 | 453.8 | 722.7 | 702.8 | 239.6 | 34.1 |

NQ: Not quantifiable. No peak or below limit of quantification (LOQ: 5.0 ng/mL)

Plasma concentrations of KB-1801 following a 24-hour intravenous infusion at 10 mg/kg to female Sprague-Dawley rats are shown in Table 19.

TABLE 19

| | | | | | | | Mean | SD | |
|---|---|---|---|---|---|---|---|---|---|
| Time | Animal ID | | | | | | (ng/ | (ng/ | % |
| (h) | 6501 | 6502 | 6503 | 6504 | 6505 | 6506 | mL) | mL) | CV |
| 1 | 361.0 | 171.3 | 115.1 | — | — | — | 215.8 | 128.9 | 59.7 |
| 2 | — | — | — | 383.4 | 236.8 | 224.0 | 281.4 | 88.6 | 31.5 |
| 4 | 445.0 | 514.4 | 318.2 | — | — | — | 425.8 | 99.5 | 23.4 |
| 6 | — | — | — | 725.9 | 517.6 | 464.6 | 569.4 | 138.1 | 24.3 |
| 8 | 678.3 | 443.8 | 454.7 | — | — | — | 525.6 | 132.4 | 25.2 |
| 24 | — | — | — | 753.1 | 955.6 | 1151.8 | 953.5 | 199.4 | 20.9 |

NQ: Not quantifiable. No peak or below limit of quantification (LOQ: 5.0 ng/mL)

Plasma concentrations of KB-1801 following a 24-hour intravenous infusion at 30 mg/kg to male Sprague-Dawley rats are shown in Table 20.

TABLE 20

| | Animal ID | | | | | | Mean | SD | |
|---|---|---|---|---|---|---|---|---|---|
| Time (h) | 7001 | 7002 | 7003 | 7004 | 7005 | 7006 | (ng/mL) | (ng/mL) | % CV |
| 1 | 953.9 | 1465.9 | 631.3 | — | — | — | 1017.0 | 420.9 | 41.4 |
| 2 | — | — | — | 918.9 | 1589.9 | 777.4 | 1095.4 | 434.0 | 39.6 |
| 4 | 1463.9 | 1178.3 | 1318.0 | — | — | — | 1320.0 | 142.8 | 10.8 |
| 6 | — | — | — | 1174.6 | 1593.6 | 1640.0 | 1469.4 | 256.3 | 17.4 |
| 8 | 1452.0 | 1580.3 | 1471.5 | — | — | — | 1501.3 | 69.1 | 4.61 |
| 24 | — | — | — | 2878.0 | 3331.2 | 2290.3 | 2833.2 | 521.9 | 18.4 |

NQ: Not quantifiable. No peak or below limit of quantification (LOQ: 5.0 ng/mL)

Plasma concentrations of KB-1801 following a 24-hour intravenous infusion at 30 mg/kg to female Sprague-Dawley rats are shown in Table 21.

TABLE 21

| | Animal ID | | | | | | Mean | SD | |
|---|---|---|---|---|---|---|---|---|---|
| Time (h) | 7501 | 7502 | 7503 | 7504 | 7505 | 7506 | (ng/mL) | (ng/mL) | % CV |
| 1 | 603.3 | 615.7 | 704.9 | — | — | — | 641.3 | 55.4 | 8.64 |
| 2 | — | — | — | 1952.9 | 1031.6 | 1309.5 | 1431.3 | 472.6 | 33.0 |
| 4 | 1248.5 | 1396.0 | 1936.4 | — | — | — | 1527.0 | 362.2 | 23.7 |
| 6 | — | — | — | 1388.6 | 1903.2 | 1640.0 | 1643.9 | 257.3 | 15.7 |
| 8 | 1739.1 | 1362.1 | 2091.5 | — | — | — | 1730.9 | 364.8 | 21.1 |
| 24 | — | — | — | 3772.1 | 1746.0 | 1995.6 | 2504.5 | 1104.8 | 44.1 |

NQ: Not quantifiable. No peak or below limit of quantification (LOQ: 5.0 ng/mL)

Plasma concentrations of KB-1801 following a 24-hour intravenous infusion at 100 mg/kg to male Sprague-Dawley rats are shown in Table 22.

TABLE 22

| | Animal ID | | | | | | Mean | SD | |
|---|---|---|---|---|---|---|---|---|---|
| Time (h) | 8001 | 8002 | 8003 | 8004 | 8005 | 8006 | (ng/mL) | (ng/mL) | % CV |
| 1 | 1531.3 | 2774.3 | 2044.9 | — | — | — | 2116.8 | 624.6 | 29.5 |
| 2 | — | — | — | 2536.6 | 4129.6 | 1612.6 | 2759.6 | 1273.2 | 46.1 |
| 4 | 3557.5 | 3882.2 | 4393.3 | — | — | — | 3944.3 | 421.4 | 10.7 |
| 6 | — | — | — | 6326.5 | 9474.0 | 4167.8 | 6656.1 | 2668.4 | 40.1 |
| 8 | 6516.6 | 5471.2 | 5325.5 | — | — | — | 5771.1 | 649.7 | 11.3 |
| 24 | — | — | — | 10652.2 | 8719.6 | 6859.8 | 8743.8 | 1896.3 | 21.7 |

NQ: Not quantifiable. No peak or below limit of quantification (LOQ: 5.0 ng/mL)

Plasma concentrations of KB-1801 following a 24-hour intravenous infusion at 100 mg/kg to female Sprague-Dawley rats are shown in Table 23.

TABLE 23

| | Animal ID | | | | | | Mean | SD | |
|---|---|---|---|---|---|---|---|---|---|
| Time (h) | 8501 | 8502 | 8503 | 8504 | 8505 | 8506 | (ng/mL) | (ng/mL) | % CV |
| 1 | 2628.2 | 1906.4 | 1597.2 | — | — | — | 2043.9 | 529.1 | 25.9 |
| 2 | — | — | — | 176.5 | 4269.2 | 2496.4 | 2314.0 | 2052.4 | 88.7 |
| 4 | 4538.7 | 5749.5 | 5331.1 | — | — | — | 5206.5 | 615.0 | 11.8 |
| 6 | — | — | — | 3545.8 | 4434.5 | 4923.9 | 4301.4 | 698.6 | 16.2 |
| 8 | 6394.7 | 5510.0 | 5078.2 | — | — | — | 5661.0 | 671.1 | 11.9 |
| 24 | — | — | — | 5592.1 | 10396.6 | 8457.1 | 8148.6 | 2417.1 | 29.7 |

NQ: Not quantifiable. No peak or below limit of quantification (LOQ: 5.0 ng/mL)

The dose proportionality and female/male ratios at C_{24 h} are shown in Table 24.

TABLE 24

| | C24h/Dose (ng/mL) | | |
|---|---|---|---|
| | Group 6 | Group 7 | Group 8 |
| | Dose | Dose | Dose |
| | Normalized | Normalized | Normalized |
| Gender | 10 mg/kg | 30 mg/kg | 100 mg/kg |
| Male | 70.3 ± 24.0 | 94.4 ± 17.4 | 87.4 ± 19.0 |
| Female | 95.3 ± 19.9 | 83.5 ± 36.8 | 81.5 ± 24.2 |
| Female/Male C₂₄ₕ Ratio | 1.36 | 0.88 | 0.93 |

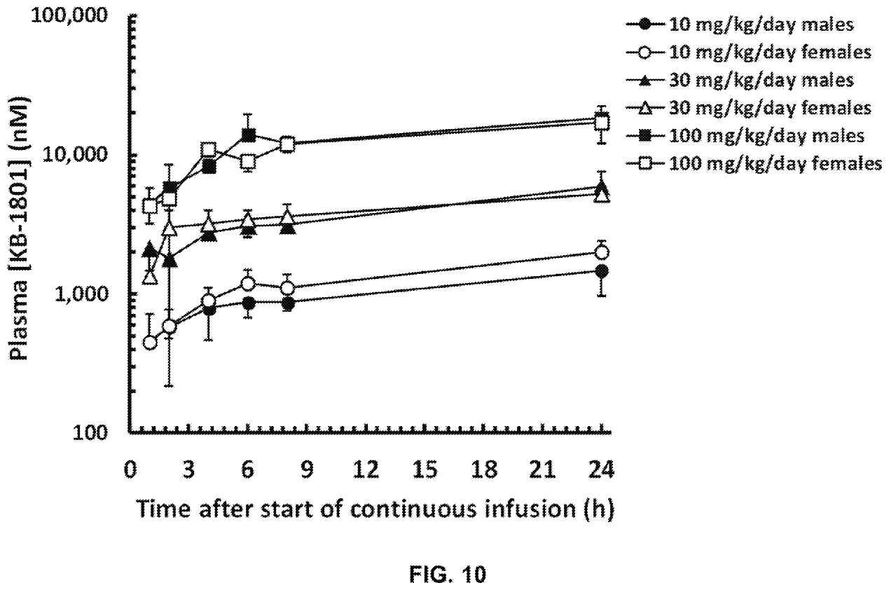

FIG. 10 is a graph of plasma PPTN concentration over time in rats during continuous intravenous administration for 24 hours. Dosing amounts are as indicated.

Plasma concentrations of KB-1801 following a 72-hour intravenous infusion at 10 mg/kg/day to male Sprague-Dawley rats are shown in Table 25.

TABLE 25

| | | | | | | | Mean | SD | |
|---|---|---|---|---|---|---|---|---|---|
| Time | Animal ID | | | | | | (ng/ | (ng/ | % |
| (h) | 14001 | 14002 | 14003 | 14004 | 14005 | 14006 | mL) | mL) | CV |
| 24 | 908.4 | 547.8 | 952.8 | — | — | — | 803.0 | 222.1 | 27.7 |
| 48 | — | — | — | 515.2 | 539.5 | 752.7 | 602.5 | 130.6 | 21.7 |
| 72 | 618.0 | 617.8 | 545.9 | — | — | — | 593.9 | 41.6 | 7.00 |
| 78 | — | — | — | 248.9 | 205.5 | 170.5 | 208.3 | 39.3 | 18.9 |
| 84 | 86.0 | 82.6 | 128.1 | — | — | — | 98.9 | 25.3 | 25.6 |
| 96 | — | — | — | 60.0 | 60.2 | 45.0 | 55.1 | 8.73 | 15.9 |

NQ: Not quantifiable. No peak or below limit of quantification (LOQ: 5.0 ng/mL)

Plasma concentrations of KB-1801 following a 72-hour intravenous infusion at 10 mg/kg/day to female Sprague-Dawley rats are shown in Table 26.

TABLE 26

| | | | | | | | Mean | SD | |
|---|---|---|---|---|---|---|---|---|---|
| Time | Animal ID | | | | | | (ng/ | (ng/ | % |
| (h) | 14501 | 14502 | 14503 | 14504 | 14505 | 14506 | mL) | mL) | CV |
| 24 | 595.1 | 784.2 | 685.7 | — | — | — | 688.3 | 94.6 | 13.7 |
| 48 | — | — | — | 721.9 | 650.5 | 588.8 | 653.8 | 66.6 | 10.2 |
| 72 | 514.3 | 756.4 | 602.0 | — | — | — | 624.2 | 122.6 | 19.6 |
| 78 | — | — | — | 150.2 | 131.4 | 160.1 | 147.2 | 14.5 | 9.87 |
| 84 | 74.8 | 103.5 | 64.3 | — | — | — | 80.9 | 20.3 | 25.1 |
| 96 | — | — | — | 29.3 | 22.9 | 15.3 | 22.5 | 6.98 | 31.0 |

NQ: Not quantifiable. No peak or below limit of quantification (LOQ: 5.0 ng/mL)

Plasma concentrations of KB-1801 following a 72-hour intravenous infusion at 30 mg/kg/day to male Sprague-Dawley rats are shown in Table 27.

TABLE 27

| | Animal ID | | | | | | Mean | SD | |
|---|---|---|---|---|---|---|---|---|---|
| Time (h) | 15001 | 15002 | 15003 | 15004 | 15005 | 15006 | (ng/mL) | (ng/mL) | % CV |
| 24 | 1515.9 | 1855.0 | 2482.4 | — | — | — | 1951.1 | 490.4 | 25.1 |
| 48 | — | — | — | 2802.1 | 2175.6 | 2510.8 | 2496.2 | 313.5 | 12.6 |
| 72 | 1105.3 | 1871.2 | 2031.5 | — | — | — | 1669.3 | 495.0 | 29.7 |
| 78 | — | — | — | 697.8 | 690.2 | 818.5 | 735.5 | 72.0 | 9.8 |
| 84 | 289.4 | 273.1 | 309.1 | — | — | — | 290.5 | 18.0 | 6.2 |
| 96 | — | — | — | 138.8 | 164.1 | 213.1 | 172.0 | 37.8 | 22.0 |

NQ: Not quantifiable. No peak or below limit of quantification (LOQ: 5.0 ng/mL)

Plasma concentrations of KB-1801 following a 72-hour intravenous infusion at 30 mg/kg/day to female Sprague-Dawley rats are shown in Table 28.

TABLE 28

| | Animal ID | | | | | | Mean | SD | |
|---|---|---|---|---|---|---|---|---|---|
| Time (h) | 15501 | 15502 | 15503 | 15504 | 15505 | 15506 | (ng/mL) | (ng/mL) | % CV |
| 24 | 2416.5 | 2027.1 | 2581.5 | — | — | — | 2341.7 | 284.7 | 12.2 |
| 48 | — | — | — | 3216.8 | 1661.0 | 1763.6 | 2213.8 | 870.1 | 39.3 |
| 72 | 2209.2 | 1397.7 | 2300.3 | — | — | — | 1969.0 | 496.9 | 25.2 |
| 78 | — | — | — | 597.4 | 449.3 | 585.0 | 543.9 | 82.1 | 15.1 |
| 84 | 453.1 | 160.2 | 306.2 | — | — | — | 306.5 | 146.5 | 47.8 |
| 96 | — | — | — | 81.6 | 75.6 | 115.3 | 90.8 | 21.4 | 23.5 |

NQ: Not quantifiable. No peak or below limit of quantification (LOQ: 5.0 ng/mL)

Plasma concentrations of KB-1801 following a 72-hour intravenous infusion at 100 mg/kg to male Sprague-Dawley rats are shown in Table 29.

TABLE 29

| | Animal ID | | | | | | Mean | SD | |
|---|---|---|---|---|---|---|---|---|---|
| Time (h) | 16001 | 16002 | 16003 | 16004 | 16005 | 16006 | (ng/mL) | (ng/mL) | % CV |
| 24 | 10336.0 | 7597.9 | 10702.8 | — | — | — | 9545.6 | 1696.7 | 17.8 |
| 48 | — | — | — | 7398.3 | 6182.8 | 7504.3 | 7028.5 | 734.3 | 10.4 |
| 72 | 11811.6 | 7768.2 | NS | — | — | — | 9789.9 | 2859.1 | 29.2 |
| 78 | — | — | — | 3223.6 | 1893.5 | 2808.6 | 2641.9 | 680.5 | 25.8 |
| 84 | 2651.1 | 1818.2 | NS | — | — | — | 2234.6 | 589.0 | 26.4 |
| 96 | — | — | — | 597.5 | 333.5 | 482.1 | 471.0 | 132.3 | 28.1 |

NQ: Not quantifiable. No peak or below limit of quantification (LOQ: 5.0 ng/mL)
NS: No Sample. Animal 16003 died shortly after the 24h timepoint according to CRL.

Plasma concentrations of KB-1801 following a 72-hour intravenous infusion at 100 mg/kg/day to female Sprague-Dawley rats are shown in Table 30.

TABLE 30

| Time | Animal ID | | | | | | Mean | SD | % |
|---|---|---|---|---|---|---|---|---|---|
| (h) | 16501 | 16502 | 16503 | 16504 | 16505 | 16506 | (ng/mL) | (ng/mL) | CV |
| 24 | 8267.8 | 6709.0 | 8238.2 | — | — | — | 7738.3 | 891.5 | 11.5 |
| 48 | — | — | — | 6070.1 | 5872.0 | 8226.5 | 6722.9 | 1305.9 | 19.4 |
| 72 | 7114.6 | 6680.1 | 6734.5 | — | — | — | 6843.1 | 236.7 | 3.46 |
| 78 | — | — | — | 1896.0 | 1454.4 | 2119.8 | 1823.4 | 338.6 | 18.6 |
| 84 | 840.6 | 1075.5 | 1046.1 | — | — | — | 987.4 | 128.0 | 13.0 |
| 96 | — | — | — | 287.7 | 211.8 | 342.6 | 280.7 | 65.7 | 23.4 |

NQ: Not quantifiable. No peak or below limit of quantification (LOQ: 5.0 ng/mL)

The calculated TK parameters following 72 hours of intravenous infusion at 10, 30 and 100 mg/kg to Sprague-Dawley rats are shown in Table 31.

TABLE 31

| | | Group14 | Group 15 | Group 16 |
|---|---|---|---|---|
| Parameter | Gender | 10 mg/kg/day | 30 mg/kg/day | 100 mg/kg/day |
| C₇₂ₕ/Dose | Male | 59.4 ± 4.16 | 55.6 ± 16.5 | 97.9 ± 28.6 |
| (ng/mL) | | | | |
| Accumulation | | 0.74 | 0.86 | 1.03 |
| ratio | | | | |
| Half-life (h) | | 7.91 ± 1.06 | 8.29 ± 0.93 | 8.83 ± 3.33 |
| Css (ng/mL) | | 636.4 ± 96.8 | 1974.4 ± 240.2 | 7774.6 ± 1241.3 |
| C₇₂ₕ/Dose | Female | 62.4 ± 12.3 | 65.6 ± 16.6 | 68.4 ± 2.37 |
| (ng/mL) | | | | |
| Accumulation | | 0.91 | 0.84 | 0.88 |
| ratio (C₇₂ₕ/C₂₄ₕ) | | | | |
| Half-life (h) | | 6.67 ± 1.21 | 7.06 ± 0.93 | 6.64 ± 0.36 |
| Css (ng/mL) | | 606.0 ± 52.0 | 2037.0 ± 433.7 | 6595.6 ± 696.2 |
| Female/ | — | 1.05 | 1.18 | 0.70 |
| Male C₇₂ₕ | | | | |

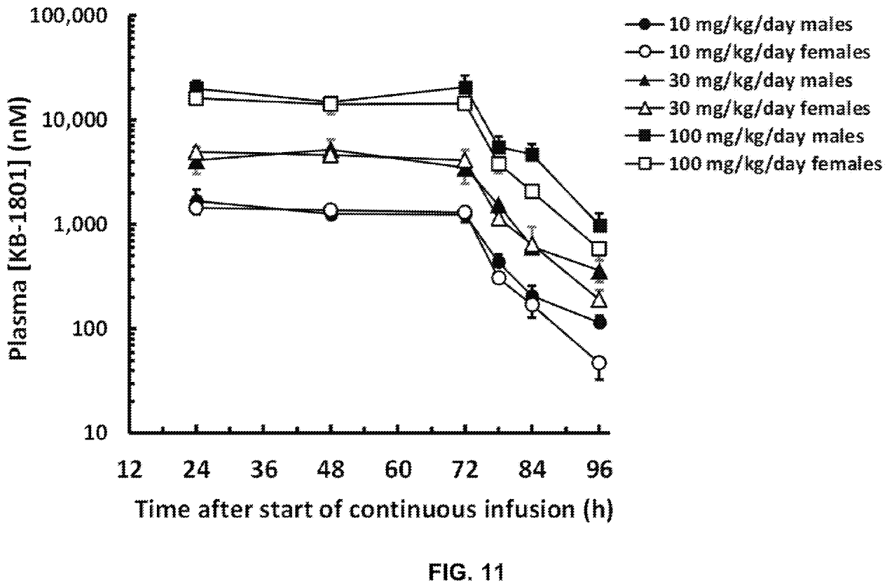

FIG. 11 is a graph of plasma PPTN concentration over time in rats during continuous intravenous administration for 72 hours. Dosing amounts are as indicated.

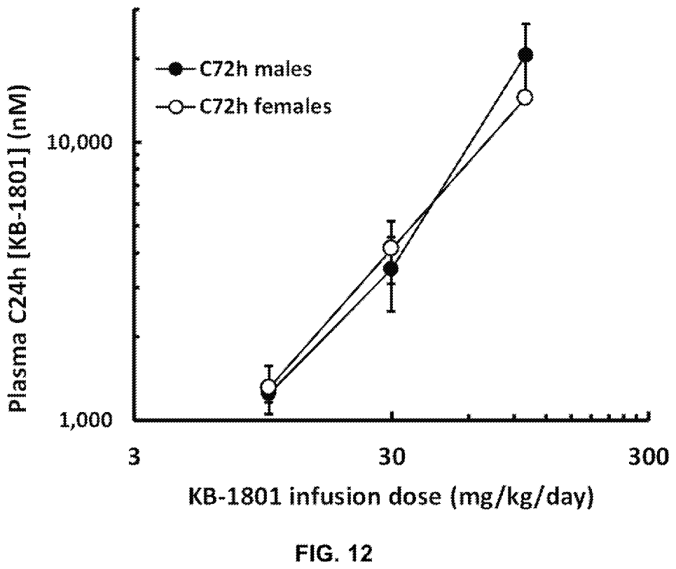

FIG. 12 is a graph of plasma PPTN concentration as a function of dose in rats during continuous intravenous infusion. Dosing amounts are as indicated.

Urine PPTN Concentrations: Results

Urine concentrations of KB-1801 following a 24-hour intravenous infusion at 10 mg/kg to male Sprague-Dawley rats are shown in Table 32.

TABLE 32

| Animal ID | Time | Conc. | Urine | Amount | Total |
|---|---|---|---|---|---|
| Body weight | Interval (h) | (μg/mL) | Volume (mL) | (μg) | amount (μg) |
| 6001 | 0-4 h | NQ | 0.50 | NC | 19.0 |
| 296 g | 4-9 h | 1.30 | 0.40 | 0.52 | |
| | 9-24 h | 37.0 | 0.50 | 18.5 | |
| 6002 | 0-4 h | 0.06 | 0.50 | 0.03 | 3.25 |
| 288 g | 4-9 h | NS | NS | NC | |
| | 9-24 h | 32.3 | 0.10 | 3.23 | |
| 6003 | 0-4 h | 5.36 | 1.00 | 5.36 | 45.5 |
| 324 g | 4-9 h | 11.2 | 0.40 | 4.47 | |
| | 9-24 h | 17.8 | 2.00 | 35.7 | |
| 6004 | 0-4 h | 1.41 | 1.50 | 2.11 | 49.8 |
| 318 g | 4-9 h | 1.16 | 0.30 | 0.35 | |
| | 9-24 h | 31.6 | 1.50 | 47.3 | |
| 6005 | 0-4 h | 10.1 | 0.20 | 2.02 | 14.8 |
| 332 g | 4-9 h | 30.7 | 0.20 | 6.15 | |
| | 9-24 h | 33.2 | 0.20 | 6.64 | |
| 6006 | 0-4 h | 7.41 | 1.50 | 11.1 | 67.4 |
| 330 g | 4-9 h | NS | NS | NC | |
| | 9-24 h | 28.1 | 2.0 | 56.3 | |

NQ: Not quantifiable. No peak or below limit of quantification (LOQ: 0.0025 pg/mL)
NS: No sample
NC: Not calculated

Urine concentrations of KB-1801 following a 24-hour intravenous infusion at 10 mg/kg to female Sprague-Dawley rats are shown in Table 33.

TABLE 33

| Animal ID | Time | Conc. | Urine | Amount | Total |
|---|---|---|---|---|---|
| Body weight | Interval (h) | (μg/mL) | Volume (mL) | (μg) | amount (μg) |
| 6501 | 0-4 h | 2.49 | 2.00 | 4.98 | 20.3 |
| 212 g | 4-9 h | 0.54 | 0.10 | 0.05 | |
| | 9-24 h | 15.3 | 1.00 | 15.3 | |
| 6502 | 0-4 h | 4.21 | 0.50 | 2.11 | 2.33 |
| 212 g | 4-9 h | 0.23 | 1.00 | 0.23 | |
| | 9-24 h | NS | NS | NC | |
| 6503 | 0-4 h | 0.33 | 0.50 | 0.16 | 6.29 |
| 215 g | 4-9 h | 3.83 | 1.50 | 5.74 | |
| | 9-24 h | 25.7 | 0.02 | 0.39 | |
| 6504 | 0-4 h | 0.47 | 0.30 | 0.14 | 1.92 |
| 195 g | 4-9 h | 13.2 | 0.10 | 1.32 | |
| | 9-24 h | 38.3 | 0.01 | 0.46 | |
| 6505 | 0-4 h | 0.81 | 1.00 | 0.81 | 6.27 |
| 212 g | 4-9 h | NS | NS | NC | |
| | 9-24 h | 18.2 | 0.30 | 5.46 | |
| 6506 | 0-4 h | NS | NS | NC | 24.6 |
| 201 g | 4-9 h | 7.48 | 1.50 | 11.2 | |
| | 9-24 h | 26.8 | 0.50 | 13.4 | |

NQ: Not quantifiable. No peak or below limit of quantification (LOQ: 0.0025 pg/mL)
NS: No sample
NC: Not calculated

Urine concentrations of KB-1801 following a 24-hour intravenous infusion at 30 mg/kg to male Sprague-Dawley rats are shown in Table 34.

TABLE 34

| Animal ID | Time | Conc. | Urine | Amount | Total |
|---|---|---|---|---|---|
| Body weight | Interval (h) | (μg/mL) | Volume (mL) | (μg) | amount (μg) |
| 7001 | 0-4 h | 33.9 | 1.00 | 33.9 | 243.3 |
| 345 g | 4-9 h | 7.87 | 1.50 | 11.8 | |
| | 9-24 h | 79.0 | 2.50 | 197.6 | |
| 7002 | 0-4 h | 0.02 | 0.50 | 0.01 | 31.4 |
| 310 g | 4-9 h | 21.8 | 1.00 | 21.8 | |
| | 9-24 h | 48.2 | 0.20 | 9.64 | |
| 7003 | 0-4 h | 0.05 | 0.10 | 0.01 | 61.7 |
| 321 g | 4-9 h | 26.0 | 1.00 | 26.0 | |
| | 9-24 h | 71.5 | 0.50 | 35.7 | |
| 7004 | 0-4 h | 2.03 | 1.00 | 2.03 | 63.3 |
| 315 g | 4-9 h | 29.2 | 1.50 | 43.8 | |
| | 9-24 h | 87.1 | 0.20 | 17.4 | |
| 7005 | 0-4 h | 0.02 | 0.50 | 0.01 | 9.64 |
| 314 g | 4-9 h | 28.2 | 0.20 | 5.65 | |
| | 9-24 h | 113.8 | 0.04 | 3.98 | |
| 7006 | 0-4 h | 0.003 | 0.50 | 0.001 | 18.1 |
| 328 g | 4-9 h | 0.01 | 0.50 | 0.003 | |
| | 9-24 h | 90.6 | 0.20 | 18.1 | |

NQ: Not quantifiable. No peak or below limit of quantification (LOQ: 0.0025 pg/mL)
NS: No sample
NC: Not calculated

Urine concentrations of KB-1801 following a 24-hour intravenous infusion at 30 mg/kg to female Sprague-Dawley rats are shown in Table 35.

TABLE 35

| Animal ID | Time | Conc. | Urine | Amount | Total |
|---|---|---|---|---|---|
| Body weight | Interval (h) | (μg/mL) | Volume (mL) | (μg) | amount (μg) |
| 7501 | 0-4 h | 1.42 | 0.50 | 0.71 | 30.9 |
| 208 g | 4-9 h | 15.1 | 1.50 | 22.6 | |
| | 9-24 h | 75.6 | 0.10 | 7.56 | |
| 7502 | 0-4 h | NS | NS | NC | 57.2 |
| 200 g | 4-9 h | 19.7 | 1.50 | 29.6 | |
| | 9-24 h | 55.3 | 0.50 | 27.6 | |
| 7503 | 0-4 h | 3.51 | 2.50 | 8.76 | 22.6 |
| 215 g | 4-9 h | 9.17 | 1.00 | 9.17 | |
| | 9-24 h | 46.5 | 0.10 | 4.65 | |
| 7504 | 0-4 h | 6.66 | 0.30 | 2.00 | 2.00 |
| 215 g | 4-9 h | NS | NS | NC | |
| | 9-24 h | NS | NS | NC | |
| 7505 | 0-4 h | 0.02 | 0.20 | 0.003 | 6.83 |
| 210 g | 4-9 h | NS | NS | NC | |
| | 9-24 h | 68.3 | 0.10 | 6.83 | |
| 7506 | 0-4 h | 17.69 | 0.40 | 7.08 | 40.2 |
| 213 g | 4-9 h | 32.4 | 0.50 | 16.2 | |
| | 9-24 h | 84.8 | 0.20 | 17.0 | |

NQ: Not quantifiable. No peak or below limit of quantification (LOQ: 0.0025 pg/mL)
NS: No sample
NC: Not calculated

Urine concentrations of KB-1801 following a 24-hour intravenous infusion at 100 mg/kg to male Sprague-Dawley rats are shown in Table 36.

TABLE 36

| Animal ID | Time | Conc. | Urine | Amount | Total |
|---|---|---|---|---|---|
| Body weight | Interval (h) | (μg/mL) | Volume (mL) | (μg) | amount (μg) |
| 8001 | 0-4 h | 40.8 | 1.00 | 40.8 | 228.4 |
| 326 g | 4-9 h | 32.1 | 1.00 | 32.1 | |
| | 9-24 h | 155.6 | 1.00 | 155.6 | |
| 8002 | 0-4 h | 128.2 | 0.40 | 51.3 | 526.5 |
| 304 g | 4-9 h | 143.1 | 0.50 | 71.5 | |
| | 9-24 h | 403.7 | 1.00 | 403.7 | |
| 8003 | 0-4 h | NQ | 0.50 | NC | 239.6 |
| 303 g | 4-9 h | 22.4 | 1.50 | 33.6 | |
| | 9-24 h | 412.0 | 0.50 | 206.0 | |
| 8004 | 0-4 h | NQ | 0.50 | NC | 82.2 |
| 316 g | 4-9 h | 52.0 | 0.50 | 26.0 | |
| | 9-24 h | 561.5 | 0.10 | 56.1 | |
| 8005 | 0-4 h | NQ | 1.00 | NC | 0.001 |
| 309 g | 4-9 h | 0.004 | 0.30 | 0.001 | |
| | 9-24 h | NS | NS | NC | |
| 8006 | 0-4 h | 0.24 | 0.50 | 0.12 | 186.8 |
| 197 g | 4-9 h | 60.3 | 0.40 | 24.1 | |
| | 9-24 h | 541.9 | 0.30 | 162.6 | |

NQ: Not quantifiable. No peak or below limit of quantification (LOQ: 0.0025 pg/mL)
NS: No sample
NC: Not calculated

Urine concentrations of KB-1801 following a 24-hour intravenous infusion at 100 mg/kg to female Sprague-Dawley rats are shown in Table 37.

TABLE 37

| Animal ID | Time | Conc. | Urine | Amount | Total |
|---|---|---|---|---|---|
| Body weight | Interval (h) | (μg/mL) | Volume (mL) | (μg) | amount (μg) |
| 8501 | 0-4 h | 17.1 | 1.50 | 25.6 | 149.9 |
| 233 g | 4-9 h | 0.01 | 1.00 | 0.01 | |
| | 9-24 h | 248.5 | 0.50 | 124.3 | |
| 8502 | 0-4 h | 2.38 | 4.50 | 10.7 | 171.6 |
| 232 g | 4-9 h | 18.3 | 1.50 | 27.4 | |
| | 9-24 h | 26.7 | 5.00 | 133.5 | |
| 8503 | 0-4 h | 0.01 | 2.00 | 0.02 | 45.8 |
| 221 g | 4-9 h | 3.67 | 0.50 | 1.83 | |
| | 9-24 h | 146.4 | 0.30 | 43.9 | |
| 8504 | 0-4 h | 0.01 | 1.00 | 0.01 | 48.8 |
| 208 g | 4-9 h | NS | NS | NC | |
| | 9-24 h | 48.8 | 1.00 | 48.8 | |
| 8505 | 0-4 h | 9.75 | 1.00 | 9.75 | 64.7 |
| 205 g | 4-9 h | 48.3 | 0.40 | 19.3 | |
| | 9-24 h | 356.5 | 0.10 | 35.7 | |
| 8506 | 0-4 h | 3.61 | 1.00 | 3.61 | 43.0 |
| 213 g | 4-9 h | 19.9 | 0.30 | 5.96 | |
| | 9-24 h | 167.0 | 0.20 | 33.4 | |

NQ: Not quantifiable. No peak or below limit of quantification (LOQ: 0.0025 pg/mL)
NS: No sample
NC: Not calculated

Urine concentrations of KB-1801 following a 72-hour intravenous infusion at 10 mg/kg/day to male Sprague-Dawley rats are shown in Table 38.

TABLE 38

| Animal ID | Time | Conc. | Urine | Amount | Total |
|---|---|---|---|---|---|
| Body weight | Interval (h) | (μg/mL) | Volume (mL) | (μg) | amount (μg) |
| 14001 | 0-4 h | 7.91 | 0.50 | 3.96 | 60.9 |
| 315 g | 4-9 h | 33.3 | 0.30 | 9.98 | |
| | 20-24 h | 20.9 | 1.00 | 20.9 | |
| | 44-48 h | 24.7 | 0.40 | 9.88 | |
| | 68-72 h | 16.1 | 1.00 | 16.1 | |
| | 92-96 h | 0.13 | 0.50 | 0.07 | |
| 14002 | 0-4 h | NS | NS | NC | 54.2 |
| 307 g | 4-9 h | 7.64 | 1.50 | 11.5 | |
| | 20-24 h | 20.7 | 1.00 | 20.7 | |
| | 44-48 h | 24.8 | 0.10 | 2.48 | |
| | 68-72 h | 19.3 | 1.00 | 19.3 | |
| | 92-96 h | 0.10 | 1.50 | 0.15 | |
| 14003 | 0-4 h | 6.09 | 1.00 | 6.09 | 46.1 |
| 335 g | 4-9 h | 5.46 | 1.00 | 5.46 | |
| | 20-24 h | 20.1 | 0.50 | 10.0 | |
| | 44-48 h | NS | NS | NC | |
| | 68-72 h | 23.2 | 1.00 | 23.2 | |
| | 92-96 h | 0.62 | 2.00 | 1.23 | |
| 14004 | 0-4 h | 8.16 | 0.50 | 4.08 | 62.5 |
| 349 g | 4-9 h | 17.0 | 0.50 | 8.50 | |
| | 20-24 h | 24.0 | 0.30 | 7.20 | |
| | 44-48 h | 14.1 | 1.00 | 14.1 | |
| | 68-72 h | 28.5 | 1.00 | 28.5 | |
| | 92-96 h | 0.20 | 0.40 | 0.08 | |
| 14005 | 0-4 h | NS | NS | NC | 83.7 |
| 352 g | 4-9 h | 6.95 | 1.00 | 6.95 | |
| | 20-24 h | 10.3 | 3.00 | 30.9 | |
| | 44-48 h | 45.9 | 0.50 | 22.9 | |
| | 68-72 h | 22.7 | 1.00 | 22.7 | |
| | 92-96 h | 0.09 | 2.00 | 0.18 | |
| 14006 | 0-4 h | 3.50 | 0.50 | 1.75 | 74.4 |
| 352 g | 4-9 h | 15.8 | 0.50 | 7.88 | |
| | 20-24 h | 19.4 | 0.50 | 9.72 | |
| | 44-48 h | 13.7 | 2.50 | 34.1 | |
| | 68-72 h | 20.9 | 1.00 | 20.9 | |
| | 92-96 h | 0.07 | 0.20 | 0.01 | |

NQ: Not quantifiable. No peak or below limit of quantification (LOQ: 0.0025 pg/mL)
NS: No sample
NC: Not calculated

Urine concentrations of KB-1801 following a 72-hour intravenous infusion at 10 mg/kg/day to female Sprague-Dawley rats are shown in Table 39.

TABLE 39

| Animal ID | Time | Conc. | Urine | Amount | Total |
|---|---|---|---|---|---|
| Body weight | Interval (h) | (μg/mL) | Volume (mL) | (μg) | amount (μg) |
| 14501 | 0-4 h | NS | NS | NC | 29.4 |
| 223 g | 4-9 h | 18.1 | 0.40 | 7.26 | |
| | 20-24 h | 13.0 | 0.50 | 6.50 | |
| | 44-48 h | 9.06 | 0.50 | 4.53 | |
| | 68-72 h | 11.1 | 1.00 | 11.1 | |
| | 92-96 h | 0.01 | 0.50 | 0.003 | |
| 14502 | 0-4 h | 1.10 | 1.00 | 1.10 | 27.3 |
| 231 g | 4-9 h | 14.7 | 0.20 | 2.94 | |
| | 20-24 h | 25.7 | 0.20 | 5.14 | |
| | 44-48 h | 11.2 | 1.50 | 16.8 | |
| | 68-72 h | 12.9 | 0.10 | 1.29 | |
| | 92-96 h | 0.10 | 0.10 | 0.01 | |
| 14503 | 0-4 h | 0.02 | 0.01 | 0.0002 | 16.4 |
| 221 g | 4-9 h | NS | NS | NC | |
| | 20-24 h | 18.5 | 0.50 | 9.24 | |
| | 44-48 h | 21.8 | 0.10 | 2.18 | |
| | 68-72 h | 4.95 | 1.00 | 4.95 | |
| | 92-96 h | 0.02 | 0.03 | 0.0005 | |
| 14504 | 0-4 h | NS | NS | NC | 10.5 |
| 199 g | 4-9 h | 14.9 | 0.10 | 1.49 | |
| | 20-24 h | 13.9 | 0.20 | 2.78 | |
| | 44-48 h | NS | NS | NC | |
| | 68-72 h | 20.7 | 0.30 | 6.21 | |
| | 92-96 h | NS | NS | NC | |
| 14505 | 0-4 h | 0.01 | 0.07 | 0.0004 | 18.5 |
| 225 g | 4-9 h | 5.69 | 1.50 | 8.54 | |
| | 20-24 h | NS | NS | NC | |
| | 44-48 h | 13.5 | 0.50 | 6.75 | |
| | 68-72 h | 31.8 | 0.10 | 3.18 | 20.9 |
| | 92-96 h | 0.01 | 0.50 | 0.004 | |
| 14506 | 0-4 h | NQ | 0.01 | NC | |
| 208 g | 4-9 h | 5.44 | 1.00 | 5.44 | |
| | 20-24 h | 20.7 | 0.30 | 6.21 | |
| | 44-48 h | 16.5 | 0.20 | 3.31 | |
| | 68-72 h | 11.8 | 0.50 | 5.92 | |
| | 92-96 h | 0.05 | 0.01 | 0.001 | |

NQ: Not quantifiable. No peak or below limit of quantification (LOQ: 0.0025 pg/mL)
NS: No sample
NC: Not calculated

Urine concentrations of KB-1801 following a 72-hour intravenous infusion at 30 mg/kg/day to male Sprague-Dawley rats are shown in Table 40.

TABLE 40

| Animal ID | Time | Conc. | Urine | Amount | Total |
|---|---|---|---|---|---|
| Body weight | Interval (h) | (μg/mL) | Volume (mL) | (μg) | amount (μg) |
| 15001 | 0-4 h | 5.72 | 0.40 | 2.29 | 332.4 |
| 373 g | 4-9 h | 73.3 | 0.40 | 29.3 | |
| | 20-24 h | 41.6 | 2.00 | 83.2 | |
| | 44-48 h | 63.5 | 2.00 | 127.0 | |
| | 68-72 h | 60.0 | 1.50 | 90.1 | |
| | 92-96 h | 0.26 | 2.00 | 0.51 | |
| 15002 | 0-4 h | 7.51 | 1.00 | 7.51 | 188.0 |
| 332 g | 4-9 h | 34.0 | 0.50 | 17.0 | |
| | 20-24 h | 70.0 | 0.50 | 35.0 | |
| | 44-48 h | 64.7 | 1.00 | 64.7 | |
| | 68-72 h | 63.2 | 1.00 | 63.2 | |
| | 92-96 h | 0.23 | 2.00 | 0.47 | |
| 15003 | 0-4 h | 32.1 | 0.06 | 1.93 | 165.1 |
| 360 g | 4-9 h | 76.7 | 0.04 | 3.07 | |
| | 20-24 h | NS | NS | NC | |
| | 44-48 h | 82.2 | 1.00 | 82.2 | |
| | 68-72 h | 77.8 | 1.00 | 77.8 | |
| | 92-96 h | 0.52 | 0.20 | 0.10 | |
| 15004 | 0-4 h | 23.9 | 0.05 | 1.19 | 250.3 |
| 333 g | 4-9 h | 20.7 | 1.50 | 31.1 | |
| | 20-24 h | 55.7 | 1.00 | 55.7 | |
| | 44-48 h | 53.1 | 2.00 | 106.2 | |
| | 68-72 h | 55.7 | 1.00 | 55.7 | |
| | 92-96 h | 0.53 | 1.00 | 0.53 | |
| 15005 | 0-4 h | NS | NS | NC | 108.4 |
| 341 g | 4-9 h | NS | NS | NC | |
| | 20-24 h | 111.5 | 0.30 | 33.4 | |
| | 44-48 h | 64.0 | 0.50 | 32.0 | |
| | 68-72 h | 85.4 | 0.50 | 42.7 | |
| | 92-96 h | 0.52 | 0.50 | 0.26 | |
| 15006 | 0-4 h | 0.89 | 0.01 | 0.0 | 87.4 |
| 348 g | 4-9 h | 114.0 | 0.10 | 11.4 | |
| | 20-24 h | 78.0 | 0.50 | 39.0 | |
| | 44-48 h | 114.6 | 0.10 | 11.5 | |
| | 68-72 h | 127.1 | 0.20 | 25.4 | |
| | 92-96 h | 0.39 | 0.20 | 0.08 | |

NQ: Not quantifiable. No peak or below limit of quantification (LOQ: 0.0025 pg/mL)
NS: No sample
NC: Not calculated

Urine concentrations of KB-1801 following a 72-hour intravenous infusion at 30 mg/kg/day to female Sprague-Dawley rats are shown in Table 41.

TABLE 41

| Animal ID | Time | Conc. | Urine | Amount | Total |
|---|---|---|---|---|---|
| Body weight | Interval (h) | (μg/mL) | Volume (mL) | (μg) | amount (μg) |
| 15501 | 0-4 h | 5.36 | 0.40 | 2.15 | 131.7 |
| 210 g | 4-9 h | 30.2 | 0.50 | 15.1 | |
| | 20-24 h | 16.0 | 1.00 | 16.0 | |
| | 44-48 h | 38.9 | 1.50 | 58.3 | |
| | 68-72 h | 20.0 | 2.00 | 40.1 | |
| | 92-96 h | 0.02 | 0.50 | 0.008 | |
| 15502 | 0-4 h | 4.94 | 0.40 | 1.98 | 75.7 |
| 223 g | 4-9 h | 33.4 | 0.30 | 10.0 | |
| | 20-24 h | 24.1 | 0.50 | 12.0 | |
| | 44-48 h | 50.5 | 0.50 | 25.2 | |
| | 68-72 h | 26.4 | 1.00 | 26.4 | |
| | 92-96 h | 0.04 | 0.20 | 0.01 | |
| 15503 | 0-4 h | 5.26 | 0.40 | 2.11 | 78.9 |
| 216 g | 4-9 h | 21.7 | 0.50 | 10.8 | |
| | 20-24 h | 27.5 | 1.00 | 27.5 | |
| | 44-48 h | 31.4 | 1.00 | 31.4 | |
| | 68-72 h | 34.8 | 0.20 | 6.96 | |
| | 92-96 h | 0.02 | 1.50 | 0.03 | |
| 15504 | 0-4 h | NS | NS | NC | 78.9 |
| 211 g | 4-9 h | 52.8 | 0.20 | 10.6 | |
| | 20-24 h | 39.3 | 0.40 | 15.7 | |
| | 44-48 h | 52.6 | 1.00 | 52.6 | |
| | 68-72 h | NS | NS | NC | |
| | 92-96 h | 0.10 | 0.06 | 0.01 | |
| 15505 | 0-4 h | NS | NS | NC | 66.9 |
| 220 g | 4-9 h | 22.6 | 1.50 | 33.8 | |
| | 20-24 h | 84.9 | 0.10 | 8.49 | |
| | 44-48 h | 45.6 | 0.30 | 13.7 | |
| | 68-72 h | 54.2 | 0.20 | 10.8 | |
| | 92-96 h | 0.11 | 0.20 | 0.02 | |
| 15506 | 0-4 h | NS | NS | NC | 80.5 |
| 233 g | 4-9 h | NS | NS | NC | |
| | 20-24 h | 66.5 | 1.00 | 66.5 | |
| | 44-48 h | 61.9 | 0.20 | 12.4 | |
| | 68-72 h | 78.3 | 0.02 | 1.57 | |
| | 92-96 h | 0.09 | 0.20 | 0.02 | |

NQ: Not quantifiable. No peak or below limit of quantification (LOQ: 0.0025 pg/mL)
NS: No sample
NC: Not calculated

Urine concentrations of KB-1801 following a 72-hour intravenous infusion at 100 mg/kg/day to male Sprague-Dawley rats are shown in Table 42.

TABLE 42

| Animal ID | Time | Conc. | Urine | Amount | Total |
|---|---|---|---|---|---|
| Body weight | Interval (h) | (μg/mL) | Volume (mL) | (μg) | amount (μg) |
| 16001 | 0-4 h | 142.4 | 0.30 | 42.7 | 438.9 |
| 371 g | 4-9 h | 79.7 | 0.10 | 8.0 | |
| | 20-24 h | 291.4 | 0.20 | 58.3 | |
| | 44-48 h | 145.7 | 1.50 | 218.6 | |
| | 68-72 h | 220.3 | 0.50 | 110.2 | |
| | 92-96 h | 1.13 | 1.00 | 1.13 | |
| 16002 | 0-4 h | 119.9 | 0.10 | 12.0 | 337.1 |
| 332 g | 4-9 h | 218.2 | 0.10 | 21.8 | |
| | 20-24 h | 205.3 | 1.00 | 205.3 | |
| | 44-48 h | 397.5 | 0.20 | 79.5 | |
| | 68-72 h | 177.9 | 0.10 | 17.8 | |
| | 92-96 h | 1.45 | 0.50 | 0.73 | |
| 16003 | 0-4 h | 33.1 | 0.20 | 6.62 | 155.1 |
| 329 g | 4-9 h | 284.7 | 0.10 | 28.5 | |
| | 20-24 h | 239.9 | 0.50 | 120.0 | |
| | 44-48 h | NS | NS | NC | |
| | 68-72 h | NS | NS | NC | |
| | 92-96 h | NS | NS | NC | |
| 16004 | 0-4 h | 110.3 | 0.10 | 11.03 | 785.2 |
| 306 g | 4-9 h | 207.1 | 0.50 | 103.5 | |
| | 20-24 h | 102.7 | 1.00 | 102.7 | |
| | 44-48 h | 200.8 | 2.00 | 401.7 | |
| | 68-72 h | 331.8 | 0.50 | 165.9 | |
| | 92-96 h | 1.68 | 0.20 | 0.34 | |
| 16005 | 0-4 h | NS | NS | NC | 453.6 |
| 335 g | 4-9 h | 135.4 | 0.50 | 67.7 | |
| | 20-24 h | 254.4 | 0.20 | 50.9 | |
| | 44-48 h | 367.0 | 0.20 | 73.4 | |
| | 68-72 h | 261.0 | 1.00 | 261.0 | |
| | 92-96 h | 1.06 | 0.50 | 0.53 | |
| 16006 | 0-4 h | 93.9 | 0.40 | 37.6 | 537.2 |
| 330 g | 4-9 h | 93.3 | 2.50 | 233.3 | |
| | 20-24 h | 265.7 | 0.20 | 53.1 | |
| | 44-48 h | 388.2 | 0.10 | 38.8 | |
| | 68-72 h | 348.8 | 0.50 | 174.4 | |
| | 92-96 h | 2.09 | 0.01 | 0.02 | |

NQ: Not quantifiable. No peak or below limit of quantification (LOQ: 0.0025 pg/mL)
NS: No sample
NC: Not calculated

Urine concentrations of KB-1801 following a 72-hour intravenous infusion at 100 mg/kg/day to female Sprague-Dawley rats are shown in Table 43.

TABLE 43

| Animal ID | Time | Conc. | Urine | Amount | Total |
|---|---|---|---|---|---|
| Body weight | Interval (h) | (μg/mL) | Volume (mL) | (μg) | amount (μg) |
| 16501 | 0-4 h | NS | NS | NC | 353.9 |
| 257 g | 4-9 h | 109.6 | 0.05 | 5.5 | |
| | 20-24 h | 124.9 | 0.40 | 50.0 | |
| | 44-48 h | 146.4 | 0.50 | 73.2 | |
| | 68-72 h | 225.2 | 1.00 | 225.2 | |
| | 92-96 h | 0.19 | 0.20 | 0.04 | |
| 16502 | 0-4 h | 31.3 | 0.06 | 1.88 | 251.8 |
| 256 g | 4-9 h | 98.8 | 0.30 | 29.6 | |
| | 20-24 h | 151.3 | 0.30 | 45.4 | |
| | 44-48 h | 79.7 | 1.50 | 119.6 | |
| | 68-72 h | 110.0 | 0.50 | 55.0 | |
| | 92-96 h | 0.41 | 0.50 | 0.21 | |
| 16503 | 0-4 h | 53.6 | 0.50 | 26.8 | 191.6 |
| 255 g | 4-9 h | 257.8 | 0.20 | 51.6 | |
| | 20-24 h | 105.5 | 0.50 | 52.8 | |
| | 44-48 h | 162.8 | 0.10 | 16.3 | |
| | 68-72 h | 220.9 | 0.20 | 44.2 | |
| | 92-96 h | 0.18 | 0.04 | 0.01 | |
| 16504 | 0-4 h | 0.01 | 0.10 | 0.0 | 78.7 |
| 220 g | 4-9 h | NS | NS | NC | |
| | 20-24 h | 0.6 | 0.10 | 0.1 | |
| | 44-48 h | NS* | NS* | NC | |
| | 68-72 h | 262.0 | 0.30 | 78.6 | |
| | 92-96 h | 0.19 | 0.50 | 0.09 | |
| 16505 | 0-4 h | NS | NS | NC | 198.3 |
| 215 g | 4-9 h | 45.7 | 1.50 | 68.6 | |
| | 20-24 h | 91.5 | 1.00 | 91.5 | |
| | 44-48 h | NS* | NS* | NC | |
| | 68-72 h | 127.6 | 0.30 | 38.3 | |
| | 92-96 h | 0.25 | 0.20 | 0.05 | |
| 16506 | 0-4 h | 0.02 | 0.01 | 0.0 | 151.4 |
| 239 g | 4-9 h | 109.3 | 0.50 | 54.7 | |
| | 20-24 h | 349.3 | 0.10 | 34.9 | |
| | 44-48 h | 175.4 | 0.30 | 52.6 | |
| | 68-72 h | 613.4 | 0.02 | 9.20 | |
| | 92-96 h | 0.10 | 0.10 | 0.01 | |

NQ: Not quantifiable. No peak or below limit of quantification (LOQ: 0.0025 pg/mL)
NS: No sample
NC: Not calculated
*Animal 16505′s 44-48h sample was collected in animal 16504′s container, contaminating both samples.
No volume was supplied.

Plasma PPTN Concentrations: Standards

Data for standard curve analysis of plasma concentrations of PPTN in rats that received continuous intravenous infusion at 10 mg/kg for 24 or 72 hours are shown in Tables 44-46.

TABLE 44

| | Curve 1 (start) | | | Curve 2 (end) | | |
|---|---|---|---|---|---|---|
| | Expected | | | Expected | | |
| | Concentration | Deviation | Accepted | Concentration | Deviation | Accepted |
| Standard | (ng/mL) | % | (±25%) | (ng/mL) | % | (±25%) |
| STD 4 | 5.00 | 17.73% | Yes | 5.00 | −16.66% | Yes |
| STD 5 | 10.00 | 1.03% | Yes | 10.00 | −41.40% | No |
| STD 6 | 25.00 | −9.09% | Yes | 25.00 | 0.28% | Yes |
| STD 7 | 50.00 | −5.80% | Yes | 50.00 | 0.66% | Yes |
| STD 8 | 100.00 | 13.48% | Yes | 100.00 | 1.34% | Yes |
| STD 9 | 250.00 | −0.93% | Yes | 250.00 | 2.26% | Yes |
| STD 10 | 500.00 | −6.40% | Yes | 500.00 | −5.53% | Yes |
| STD 11 | 1000.00 | 3.07% | Yes | 1000.00 | 7.86% | Yes |
| STD 12 | 2500.00 | −3.09% | Yes | 2500.00 | 2.68% | Yes |
| STD 13 | 5000.00 | −1.55% | Yes | 5000.00 | −3.58% | Yes |
| STD 14 | 10 000 | 3.00% | Yes | 10 000 | −0.65% | Yes |

TABLE 45

| | Expected | | | | | | |
|---|---|---|---|---|---|---|---|
| Quality | Concentration | Begin- | % | | % | | % |
| Control | (ng/mL) | ning | Deviation | Middle | Deviation | End | Deviation |
| QC low | 25.00 | 23.90 | −4.40% | 22.62 | −9.52% | 23.30 | −6.78% |
| QC med | 100.00 | 82.56 | −17.44% | 98.93 | −1.07% | 101.38 | 1.38% |
| QC high | 1000.00 | 1019.62 | 1.96% | 1091.75 | 9.17% | 1056.81 | 5.68% |

TABLE 46

| | |
|---|---|
| Curve Range | 5.0-10 000 ng/mL |
| Regression model | Quadratic |
| Weighting | 1/X² |
| Intercept | |
| Slope | |
| a (x2 quadratic) | 0.0000 |
| b (x quadratic) | 0.0001 |
| c (constant quadratic) | −0.0001 |
| Correlation (r2) | 0.9932 |
| Internal Standard used | Glyburide |

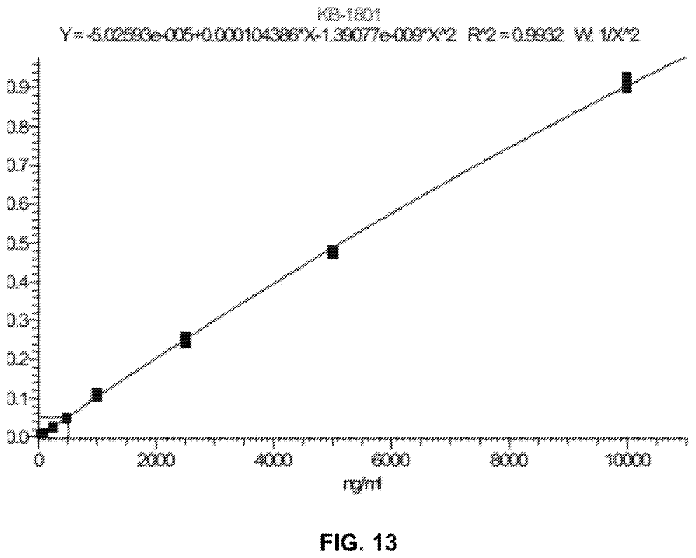

FIG. 13 is a standard curve ofplasma PPTN concentrations from rats that received continuous intravenous infusion at 10 mg/kg for 24 or72 hours. The standard curve is described by the equation Y=−0.000158985+0.000124839*X−1.72745e⁻⁰⁰⁹*X², R²=0.9879.

Assessment of bioanalytical acceptance criteria ofplasma PPTN concentrations from rats that received continuous intravenous infusion at 10 mg/kg for 24 or 72 hours is provided in Table 47.

TABLE 47

| Criteria | Value | Comment |
|---|---|---|
| Correlation Coefficient (r) > 0.99 | 0.9966 | Acceptable |
| Minimum 8 STD (±25%) used for both curves | 10 | Acceptable |
| No more than 2 STD rejected in a row | No | Acceptable |
| No more than 33% of QCs >25% of the nominal, | No | Acceptable |
| and no more than 50% of QCs per level >25% | | |
| of their nominal | | |
| Carryover after ULOQ (% of ULOQ) | 0.32% | N/AP |
| Carryover before Samples (Less than 30% of | <12.24% | Acceptable |
| LLOQ) | | |
| Response in Blank (Peak Area less than | <2.04% | Acceptable |
| 30% of LLOQ) | | |
| Response in STD 0 (Peak Area ratio less | | Acceptable |
| than 30% | | |
| of LLOQ) | <3.84% | Acceptable |
| IS sample area % cv <25% | 3.50% | |
| IS sample area outside 50%-180% of mean IS | | |
| response | None | Acceptable |
| System control response % cv < 25% | 6.45% | Acceptable |

Data for standard curve analysis of plasma concentrations of PPTN in rats that received continuous intravenous infusion at 30 mg/kg for 24 or 72 hours are shown in Tables 48-50.

TABLE 48

| | Curve 1 (start) | | | Curve 2 (end) | | |
|---|---|---|---|---|---|---|
| | Expected | | | Expected | | |
| | Concentration | Deviation | Accepted | Concentration | Deviation | Accepted |
| Standard | (ng/mL) | % | (±25%) | (ng/mL) | % | (±25%) |
| STD 4 | 5.00 | 10.86% | Yes | 5.00 | −6.30% | Yes |
| STD 5 | 10.00 | 12.07% | Yes | 10.00 | −17.76% | Yes |
| STD 6 | 25.00 | −6.32% | Yes | 25.00 | −4.55% | Yes |
| STD 7 | 50.00 | 13.56% | Yes | 50.00 | −11.27% | Yes |
| STD 8 | 100.00 | 6.00% | Yes | 100.00 | 0.46% | Yes |
| STD 9 | 250.00 | 5.51% | Yes | 250.00 | −14.95% | Yes |
| STD 10 | 500.00 | 11.59% | Yes | 500.00 | −4.17% | Yes |
| STD 11 | 1000.00 | 10.25% | Yes | 1000.00 | −5.58% | Yes |
| STD 12 | 2500.00 | 9.46% | Yes | 2500.00 | −8.93% | Yes |
| STD 13 | 5000.00 | 8.85% | Yes | 5000.00 | −6.12% | Yes |
| STD 14 | 10 000 | 9.28% | Yes | 10 000 | −11.45% | Yes |

TABLE 49

| | Expected | | | | | | |
|---|---|---|---|---|---|---|---|
| Quality | Concentration | Begin- | % | | % | | % |
| Control | (ng/mL) | ning | Deviation | Middle | Deviation | End | Deviation |
| QC low | 25.00 | 23.88 | −4.49% | 26.83 | 7.30% | 29.20 | 16.81% |
| QC med | 100.00 | 99.37 | −0.63% | 98.48 | −1.52% | 107.37 | 7.37% |
| QC high | 1000.00 | 972.39 | −2.76% | 1026.77 | 2.68% | 1010.44 | 1.04% |

TABLE 50

| | |
|---|---|
| Curve Range | 5.0-10,000 ng/mL |
| Regression model | Quadratic |
| Weighting | 1/X² |
| Intercept | |
| Slope | |
| a (x2 quadratic) | 0.0000 |
| b (x quadratic) | 0.0001 |
| c (constant quadratic) | −0.0002 |
| Correlation (r2) | 0.9879 |
| Internal Standard used | Glyburide |

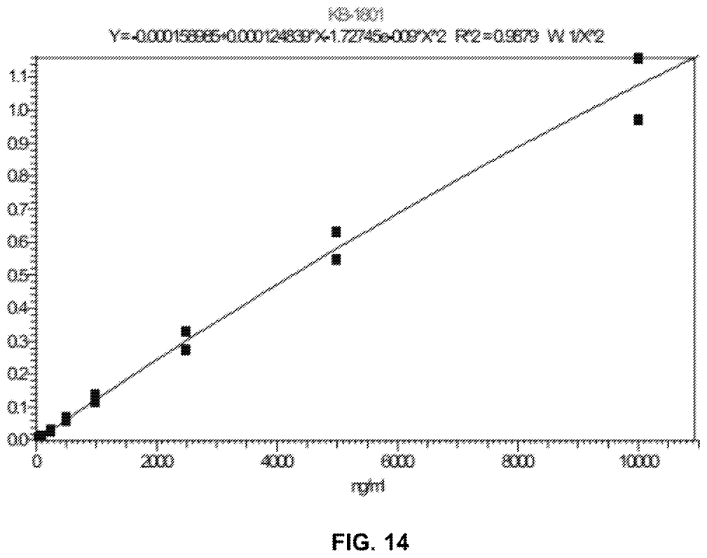

FIG. 14 is a standard curve of plasma PPTN concentrations from rats that received PP7, continuous intravenous infusion at 30 mg/kg for 24 or 72 hours. The standard curve is described by the equation Y=−0.000158985+0.000124839*X−1.72745e⁻⁰⁰⁹*X², R²=0.9879

Assessment of bioanalytical acceptance criteria of plasma PPTN concentrations from rats that received continuous intravenous infusion at 30 mg/kg for 24 or 72 hours is provided in Table 51.

TABLE 51

| Criteria | Value | Comment |
|---|---|---|
| Correlation Coefficient (r) > 0.99 | 0.9939 | Acceptable |
| Minimum 8 STD (±25%) used for both curves | 11 | Acceptable |
| No more than 2 STD rejected in a row | No | Acceptable |
| No more than 33% of QCs >25% of the | No | Acceptable |
| nominal, and no more than 50% of QCs per | | |
| level >25% of their nominal | | |
| Carryover after ULOQ (% of ULOQ) | 0.46% | N/AP |
| Carryover before Samples (Less than 30% of | <15.85% | Acceptable |
| LLOQ) | | |
| Response in Blank(Peak Area less than 30% | <9.36% | Acceptable |
| of LLOQ) | | |
| Response in STD 0 (Peak Area ratio less than | <12.65% | Acceptable |
| 30% ofLLOQ) | | |
| IS sample area % cv <25% | 4.48% | Acceptable |
| IS sample area outside 50%-180% of mean IS | None | Acceptable |
| response | | |
| System control response % cv < 25% | 11.33% | Acceptable |

Data for standard curve analysis of plasma concentrations of PPTN in rats that received continuous intravenous infusion at 100 mg/kg for 24 or 72 hours are shown in Tables 52-54.

TABLE 52

| | Curve 1 (start) | | | Curve 2 (end) | | |
|---|---|---|---|---|---|---|
| | Expected | | | Expected | | |
| | Concentration | Deviation | Accepted | Concentration | Deviation | Accepted |
| Standard | (ng/mL) | % | (±25%) | (ng/mL) | % | (±25%) |
| STD 4 | 5.00 | −5.63% | Yes | 5.00 | 2.20% | Yes |
| STD 5 | 10.00 | 6.40% | Yes | 10.00 | −5.53% | Yes |
| STD 6 | 25.00 | 6.42% | Yes | 25.00 | 1.61% | Yes |
| STD 7 | 5S0.00 | 1.34% | Yes | 50.00 | 16.91% | Yes |
| STD 8 | 100.00 | −7.39% | Yes | 100.00 | 4.81% | Yes |
| STD 9 | 250.00 | −2.15% | Yes | 250.00 | −14.78% | Yes |
| STD 10 | 500.00 | 8.54% | Yes | 500.00 | 0.93% | Yes |
| STD 11 | 1000.00 | −0.56% | Yes | 1000.00 | −6.78% | Yes |
| STD 12 | 2500.00 | −3.05% | Yes | 2500.00 | −9.08% | Yes |
| STD 13 | 5000.00 | 2.90% | Yes | 5000.00 | −0.04% | Yes |
| STD 14 | 10 000 | 2.25% | Yes | 10 000 | 1.34% | Yes |

TABLE 53

| | Expected | | | | | | |
|---|---|---|---|---|---|---|---|
| Quality | Concentration | Begin- | % | | % | | % |
| Control | (ng/mL) | ning | Deviation | Middle | Deviation | End | Deviation |
| QC low | 25.00 | 24.66 | −1.34% | 23.81 | −4.78% | 20.51 | −17.97% |
| QC med | 100.00 | 96.59 | −3.41% | 98.0. | −1.97% | 83.53 | −16.47% |
| QC high | 1000.00 | 907.55 | −9.24% | 844.69 | −15.53% | 858.92 | −14.11% |

TABLE 54

| | |
|---|---|
| Curve Range | 5.0-10,000 ng/mL |
| Regression model | Quadratic |
| Weighting | 1/X² |
| Intercept | |
| Slope | |
| a (x2 quadratic) | 0.0000 |
| b (x quadratic) | 0.0001 |
| c (constant quadratic) | −0.0002 |
| Correlation (r2) | 0.9939 |
| Internal Standard used | Glyburide |

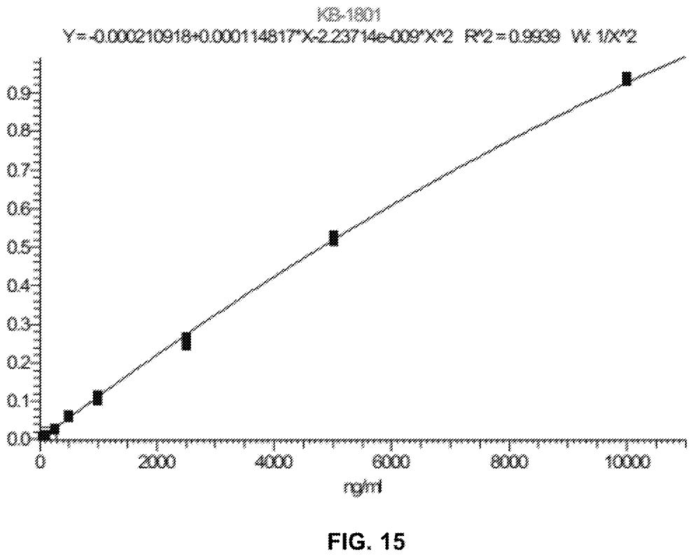

FIG. 15 is a standard curve of plasma PPTN concentrations from rats that received continuous intravenous infusion at 100 mg/kg for 24 or 72 hours. The standard curve is described by the equation Y=−0.000210918+0.0001 14817*X−2.23714e⁻⁰⁰⁹*X², R²=0.9939.

Assessment of bioanalytical acceptance criteria of plasma PPTN concentrations from rats that received continuous intravenous infusion at 100 mg/kg for 24 or 72 hours is provided in Table 55.

TABLE 55

| Criteria | Value | Comment |
|---|---|---|
| Correlation Coefficient (r) > 0.99 | 0.9969 | Acceptable |
| Minimum 8 STD (±25%) used for both curves | 11 | Acceptable |
| No more than 2 STD rejected in a row | No | Acceptable |
| No more than 33% of QCs >25% of the | No | Acceptable |
| nominal, and no more than 50% of | | |
| QCs per level >25% of their nominal | | |
| Carryover after ULOQ (% of ULOQ) | 0.29% | N/AP |
| Carryover before Samples (Less than 30% of | <20.23% | Acceptable |
| LLOQ) | | |
| Response in Blank(Peak Area less than | <5.50% | Acceptable |
| 30% of LLOQ) | | |
| Response in STD 0 (Peak Area ratio less than | <18.33% | Acceptable |
| 30% ofLLOQ) | | |
| IS sample area % cv <25% | 5.18% | Acceptable |
| IS sample area outside 50%-180% of mean IS | None | Acceptable |
| response | | |
| System control response % cv < 25% | 10.09% | Acceptable |

Urine PPTN Concentrations: Standards

Data for standard curve analysis of urine concentrations of PPTN in rats that received continuous intravenous infusion at 10 mg/kg for 24 or 72 hours are shown in Tables 56-58.

TABLE 56

| | Curve 1 (start) | | | Curve 2 (end) | | |
|---|---|---|---|---|---|---|
| | Expected | | | Expected | | |
| | Concentration | Deviation | Accepted | Concentration | Deviation | Accepted |
| Standard | (ng/mL) | % | (±25%) | (ng/mL) | % | (±25%) |
| STD 2 | 0.05 | −5.44% | Yes | 0.05 | 22.23% | Yes |
| STD 3 | 0.20 | −4.10% | Yes | 0.20 | −2.65% | Yes |
| STD 4 | 0.50 | −11.41% | Yes | 0.50 | −18.62% | Yes |
| STD 5 | 1.00 | 15.75% | Yes | 1.00 | 2.84% | Yes |
| STD 6 | 2.00 | 4.10% | Yes | 2.00 | −10.28% | Yes |
| STD 7 | 5.00 | 4.48% | Yes | 5.00 | −4.95% | Yes |
| STD 8 | 10.00 | 30.12% | No | 10.00 | 12.31% | Yes |
| STD 9 | 20.00 | 10.90% | Yes | 20.00 | 5.60% | Yes |
| STD 10 | 50.00 | 3.73% | Yes | 50.00 | −0.75% | Yes |
| STD 11 | 100.00 | −3.06% | Yes | 100.00 | −11.43% | Yes |

TABLE 57

| | Expected | | | | | | |
|---|---|---|---|---|---|---|---|
| Quality | Concentration | Begin- | % | | % | | % |
| Control | (ng/mL) | ning | Deviation | Middle | Deviation | End | Deviation |
| QC low | 1.00 | 1.04 | 3.97% | 0.96 | −4.47% | 1.05 | 5.46% |
| QC med | 10.00 | 10.97 | 9.73% | 10.46 | 4.59% | 9.95 | −0.48% |
| QC high | 100.00 | 94.92 | −5.08% | 87.45 | −12.55% | 85.38 | −14.62% |

TABLE 58

| | |
|---|---|
| Curve Range | 0.05-100.00 μg/mL |
| Regression model | Quadratic Log-Log |
| Weighting | 1/X² |
| Intercept | |
| Slope | |
| a (x2 quadratic) | |
| b (x quadratic) | |
| c (constant quadratic) | |
| Correlation (r2) | 0.9900 |
| Internal Standard used | Glyburide |

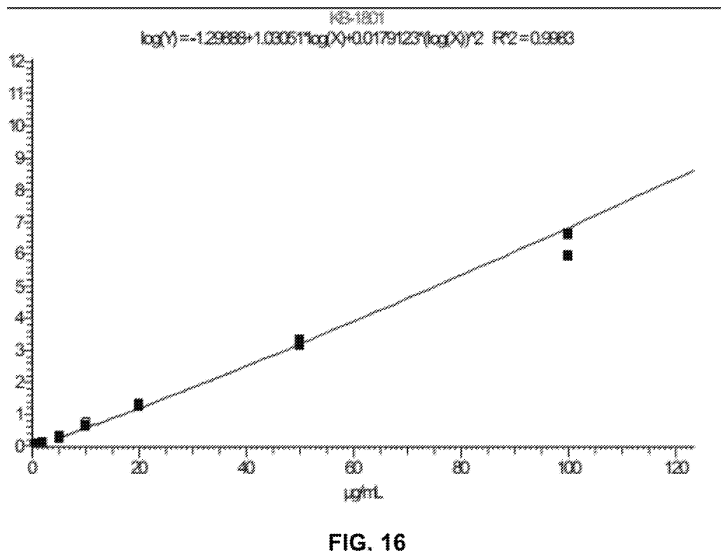

FIG. 16 is a standard curve of urine PPTN concentrations from rats that received continuous intravenous infusion at 10 mg/kg for 24 or 72 hours. The standard curve is described by the equation Y=−0.000176376+0.0595859*X−3.4615)e⁰¹*X², R²=0.9900.

Assessment of bioanalytical acceptance criteria of urine PPTN concentrations from rats that received continuous intravenous infusion at 10 mg/kg for 24 or 72 hours is provided in Table 59.

TABLE 59

| Criteria | Value | Comment |
|---|---|---|
| Correlation Coefficient (r) > 0.99 | 0.9950 | Acceptable |
| Minimum 8 STD (±25%) used for both curves | 9 | Acceptable |
| No more than 2 STD rejected in a row | No | Acceptable |
| No more than 33% of QCs >25% of the | No | Acceptable |
| nominal, and no more than 50% of QCs | | |
| per level >25% of their nominal | | |
| Carryover after ULOQ (% of ULOQ) | 0.15% | N/AP |
| Carryover before Samples (Less than 30% of | <5.22 | % Acceptable |
| LLOQ) | | |
| Response in Blank(Peak Area less than 30% | <10.80% | Acceptable |
| of LLOQ) | | |
| Response in STD 0 (Peak Area ratio less than | <7.45% | Acceptable |
| 30% ofLLOQ) | | |
| IS sample area % cv <25% | 10.08% | Acceptable |
| IS sample area outside 50%-180% of mean IS | | Acceptable |
| response | | |
| System control response % cv < 25% | 5.80% | Acceptable |

Data for standard curve analysis of urine concentrations of PPTN in rats that received continuous intravenous infusion at 30 mg/kg for 24 or 72 hours are shown in Tables 60-62.

TABLE 60

| | Curve 1 (start) | | | Curve 2 (end) | | |
|---|---|---|---|---|---|---|
| | Expected | | | Expected | | |
| | Concentration | Deviation | Accepted | Concentration | Deviation | Accepted |
| Standard | (ng/mL) | % | (±25%) | (ng/mL) | % | (±25%) |
| STD 3 | 0.20 | 5.23% | Yes | 0.20 | 7.22% | Yes |
| STD 4 | 0.50 | −18.08% | Yes | 0.50 | −17.49% | Yes |
| STD 5 | 1.00 | 7.12% | Yes | 1.00 | 9.13% | Yes |
| STD 6 | 2.00 | −10.27% | Yes | 2.00 | −5.55% | Yes |
| STD 7 | 5.00 | −0.12% | Yes | 5.00 | −2.12% | Yes |
| STD 8 | 10.00 | −1.71% | Yes | 10.00 | 6.92% | Yes |
| STD 9 | 20.00 | −2.14% | Yes | 20.00 | 3.92% | Yes |
| STD 10 | 50.00 | 3.14% | Yes | 50.00 | 11.77% | Yes |
| STD 11 | 100.00 | 5.54% | Yes | 100.00 | 2.36% | Yes |
| STD 12 | 200.00 | −1.00% | Yes | 200.00 | 0.40% | Yes |
| STD 13 | 500.00 | −2.02% | Yes | 500.00 | −3.49% | Yes |

TABLE 61

| | Expected | | | | | | |
|---|---|---|---|---|---|---|---|
| Quality | Concentration | Begin- | % | | % | | % |
| Control | (ng/mL) | ning | Deviation | Middle | Deviation | End | Deviation |
| 1.00 | 1.00 | 0.97 | −2.63% | 1.17 | 17.25% | 1.07 | 7.43% |
| QC med | 10.00 | 10.05 | 0.48% | 10.97 | 9.71% | 10.74 | 7.38% |
| QC high | 100.00 | 100.51 | 0.51% | 115.02 | 15.02% | 115.90 | 15.90% |

TABLE 62

| Curve Range | 0.2-500.00 μg/mL |
|---|---|
| Regression model | Quadratic |
| Weighting | 1/X² |
| Intercept | |
| Slope | |
| a (x2 quadratic) | 0.0000 |
| b (x quadratic) | 0.0604 |
| c (constant quadratic) | −0.0038 |
| Correlation (r2) | 0.9920 |
| Internal Standard used | 0.0000 |

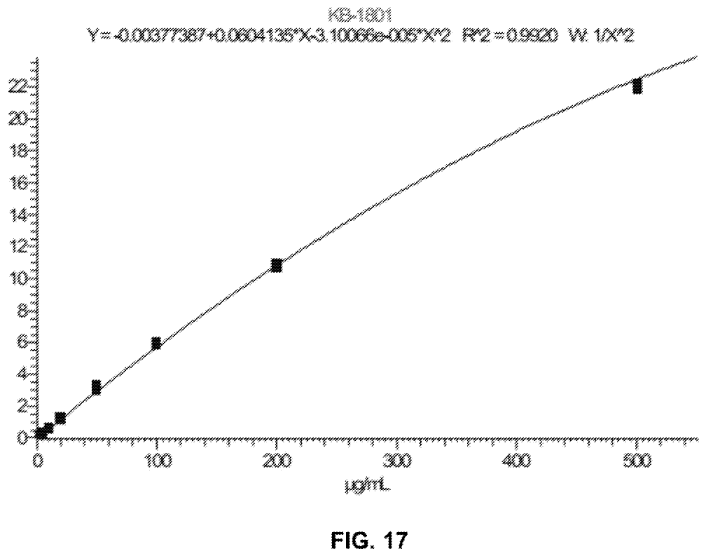

FIG. 17 is a standard curve of urine PPTN concentrations from rats that received continuous intravenous infusion at 30 mg/kg for 24 or 72 hours. The standard curve is described by the equation Y=−0.00377387+0.0604135*X−3.10066e⁻⁰⁰⁵*X², R²=0.9920.

Assessment of bioanalytical acceptance criteria of urine PPTN concentrations from rats that received continuous intravenous infusion at 30 mg/kg for 24 or 72 hours is provided in Table 63.

TABLE 63

| Criteria | Value | Comment |
|---|---|---|
| Correlation Coefficient (r) > 0.99 | 0.9960 | Acceptable |
| Minimum 8 STD (±25%) used for both curves | 11 | Acceptable |
| No more than 2 STD rejected in a row | No | Acceptable |
| No more than 33% of QCs >25% of the | No | Acceptable |
| nominal, and no more than 50% of QCs | | |
| per level >25% of their nominal | | |
| Carryover after ULOQ (% of ULOQ) | 0.07% | N/AP |
| Carryover before Samples (Less than 30% of | <1.91% | Acceptable |
| LLOQ) | | |
| Response in Blank(Peak Area less than 30% | <2.17% | Acceptable |
| of LLOQ) | | |
| Response in STD 0 (Peak Area ratio less than | <5.17% | Acceptable |
| 30% ofLLOQ) | | |
| IS sample area % cv <25% | 4.71% | Acceptable |
| IS sample area outside 50%-180% of mean IS | No | Acceptable |
| response | | |
| System control response % cv < 25% | 2.66% | Acceptable |

Data for standard curve analysis of urine concentrations of PPTN in rats that received continuous intravenous infusion at 100 mg/kg for 24 or 72 hours are shown in Tables 64-66.

TABLE 64

| | Curve 1 (start) | | | Curve 2 (end) | | |
|---|---|---|---|---|---|---|
| | Expected | | | Expected | | |
| | Concentration | Deviation | Accepted | Concentration | Deviation | Accepted |
| Standard | (ng/mL) | % | (±25%) | (ng/mL) | % | (±25%) |
| STD 2 | 0.05 | 6.71% | Yes | 0.05 | −3.41% | Yes |
| STD 3 | 0.20 | −4.83% | Yes | 0.20 | 0.99% | Yes |
| STD 4 | 0.50 | −21.37% | Yes | 0.50 | −10.56% | Yes |
| STD 5 | 1.00 | −0.45% | Yes | 1.00 | 14.42% | Yes |
| STD 6 | 2.00 | −2.46% | Yes | 2.00 | 9.06% | Yes |
| STD 7 | 5.00 | −14.64% | Yes | 5.00 | −0.33% | Yes |
| STD 8 | 10.00 | 11.92% | Yes | 10.00 | 11.95% | Yes |
| STD 9 | 20.00 | −0.61% | Yes | 20.00 | 6.86% | Yes |
| STD 10 | 50.00 | −6.53% | Yes | 50.00 | 5.66% | Yes |
| STD 11 | 100.00 | 0.32% | Yes | 100.00 | 12.07% | Yes |
| STD 12 | 200.00 | −15.22% | Yes | 200.00 | −6.55% | Yes |
| STD 13 | 500.00 | −0.18% | Yes | 500.00 | 10.87% | Yes |

TABLE 65

| | Expected | | | | | | |
|---|---|---|---|---|---|---|---|
| Quality | Concentration | Begin- | % | | % | | % |
| Control | (ng/mL) | ning | Deviation | Middle | Deviation | End | Deviation |
| QC low | 1.00 | 0.95 | −5.09% | 1.12 | 12.40% | 1.20 | 20.22% |
| QC med | 10.00 | 10.72 | 7.17% | 12.19 | 21.91% | 11.89 | 18.86% |
| QC high | 100.00 | 93.52 | −6.48% | 108.62 | 8.62% | 111.04 | 11.04% |

TABLE 66

| | |
|---|---|
| Curve Range | 0.5-500.00 μg/mL |
| Regression model | Quadratic |
| Weighting | 1/X² |
| Intercept | |
| Slope | |
| a (x2 quadratic) | 0.0000 |
| b (x quadratic) | 0.0596 |
| c (constant quadratic) | −0.0002 |
| Correlation (r2) | 0.9900 |
| Internal Standard used | Glyburide |

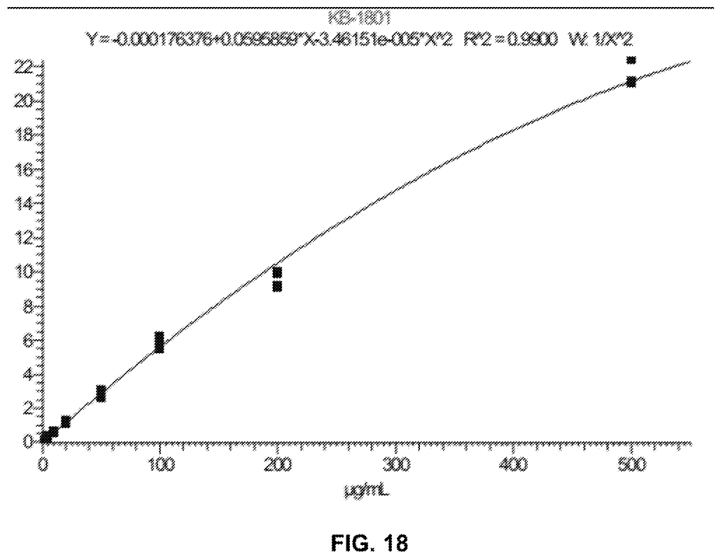

FIG. 18 is a standard curve of urine PPTN concentrations from rats that received continuous intravenous infusion at 100 mg/kg for 24 or 72 hours. The standard curve is described by the equation Y=−0.000176376+0.0595859*X−3.4615e⁻⁰⁰⁵*X², R²=0.9900.

Assessment of bioanalytical acceptance criteria of urine PPTN concentrations from rats that received continuous intravenous infusion at 100 mg/kg for 24 or 72 hours is provided in Table 67.

TABLE 67

| Criteria | Value | Comment |
|---|---|---|
| Correlation Coefficient (r) > 0.99 | 0.9950 | Acceptable |
| Minimum 8 STD (±25%) used for both | 12 | Acceptable |
| curves | | |
| No more than 2 STD rejected in a row | No | Acceptable |
| No more than 33% of QCs >25% of the | No | Acceptable |
| nominal, and no more | | |
| Carryover after ULOQ (% of ULOQ) | 0.10% | N/AP |
| Carryover before Samples (Less than 30% | <11.38 | Acceptable |
| of Response in Blank(Peak Area less than | <1.38% | Acceptable |
| 30% of LLOQ) | | |
| Response in STD 0 (Peak Area ratio less | <9.01% | Acceptable |
| than 30% ofLLOQ) | | |
| IS sample area % cv <25% | 11.38% | Acceptable |
| IS sample area outside 50%-180% of | 1 | repeat the sample |
| mean IS response | | in the next run |
| System control response % cv < 25% | 2.57% | Acceptable |

Data for standard curve analysis of a summary of urine concentrations of PPTN in rats that received continuous intravenous infusion for 24 or 72 hours are shown in Tables 68-70.

TABLE 68

| | Curve 1 (start) | | | Curve 2 (end) | | |
|---|---|---|---|---|---|---|
| Standard | Expected | Deviation | Accepted | Expected | Deviation | Accepted |
| STD 3 | 2.50 | −8.66% | Yes | 2.50 | 7.40% | Yes |
| STD 4 | 5.00 | −2.53% | Yes | 5.00 | 6.77% | Yes |
| STD 5 | 10.00 | 1.47% | Yes | 10.00 | 1.70% | Yes |
| STD 6 | 25.00 | −13.64% | Yes | 25.00 | −0.60% | Yes |
| STD 7 | 50.00 | −14.99% | Yes | 50.00 | −4.88% | Yes |
| STD 8 | 100.00 | 9.39% | Yes | 100.00 | 22.40% | Yes |
| STD 9 | 250.00 | −3.87% | Yes | 250.00 | −0.93% | Yes |
| STD 10 | 500.00 | −5.87% | Yes | 500.00 | 5.75% | Yes |
| STD 11 | 1000.00 | 2.27% | Yes | 1000.00 | −0.52% | Yes |
| STD 12 | 2500.00 | 0.71% | Yes | 2500.00 | 7.96% | Yes |
| STD 13 | 5000.00 | −0.79% | Yes | 5000.00 | 1.98% | Yes |
| STD 14 | 10 000 | −5.43% | Yes | 10 000 | −5.11% | Yes |
| STD 14 | 10 000 | −5.43% | Yes | 10 000 | −5.11% | Yes |

TABLE 69

| | Expected | | | | | | |
|---|---|---|---|---|---|---|---|
| Quality | Concentration | Begin- | % | | % | | % |
| Control | (ng/mL) | ning | Deviation | Middle | Deviation | End | Deviation |
| QC low | 25.00 | 20.10 | −19.62% | 21.96 | −12.16% | 23.25 | −7.01% |
| QC med | 100.00 | 94.24 | −5.76% | 107.83 | 7.83% | 100.95 | 0.95% |
| QC high | 1000.00 | 895.18 | −10.48% | 934.85 | −6.52% | 1085.29 | 8.53% |

TABLE 70

| | |
|---|---|
| Curve Range | 2.5-100000 ng/ml |
| Regression model | Linear |
| Weighting | 1/X² |
| Intercept | −0.00003 |
| Slope | 0.0005 |
| a (x2 quadratic) | |
| b (x quadratic) | |
| c (constant quadratic) | |
| Correlation (r2) | 0.9923 |
| Internal Standard used | Glyburide |

Summaries

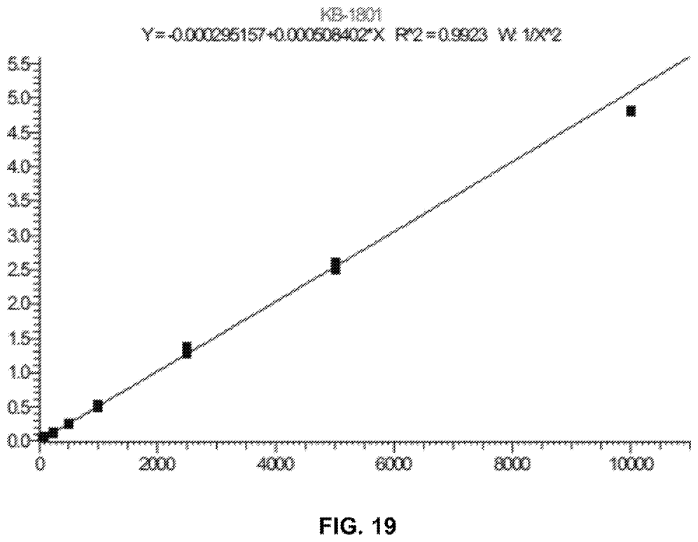

FIG. 19 is a standard curve of of a summary of urine concentrations of PPTN in rats that received continuous intravenous infusion for 24 or 72 hours. The standard curve is described by the equation Y=−0.000295157+0.000508402*X², R²=0.9923.

Assessment of bioanalytical acceptance criteria of a summary of urine concentrations of PPTN in rats that received continuous intravenous infusion for 24 or 72 hours is provided in Table 71.

TABLE 71

| Criteria | Value | Comment |
|---|---|---|
| Correlation Coefficient (r) > 0.99 | 0.9961 | Acceptable |
| Minimum 8 STD (±25%) used for both curves | 12 | Acceptable |
| No more than 2 STD rejected in a row | No | Acceptable |
| No more than 33% of QCs >25% of the | No | Acceptable |
| nominal, and no more than 50% of QCs per | | |
| level >25% of their nominal | | |
| Carryover after ULOQ (% of ULOQ) | 0.10% | N/AP |
| Carryover before Samples (Less than 30% of | <1.05% | Acceptable |
| LLOQ) | | |
| Response in Blank(Peak Area less than | <11.28% | Acceptable |
| 30% of LLOQ) | | |
| Response in STD 0 (Peak Area ratio less than | <11.39% | Acceptable |
| 30% ofLLOQ) | | |
| IS sample area % cv <25% | 12.34% | Acceptable |
| IS sample area outside 50%-180% of mean IS | No | Acceptable |
| response | | |
| System control response % cv < 25% | 8.54% | Acceptable |

A summary of the plasma analytical method is provided in Table 72.

TABLE 72
3. Analytical Method Summary Plasma
Study Summary

| | |
|---|---|
| Study Number | PK-2019-089 |
| Compound(s) | KB-1801 |
| Matrix | Rat Plasma |
| Analysis Date (s) | 27 Mar. 2019 |
| LC/MS System | Quantum 2 |

Sample Preparation

| | |
|---|---|
| Extraction Method | Protein precipitation |
| Procedure | 1. 10 pL sample + 10 pL of 0.5% formic acid in water + 180 pL |
| | internal standard working solution (0.1% FA in 0.5 pM |
| | Glyburide/Labetalol in 0.5% Ammonium formate in Methanol/ACN) |
| | 2. Vortex and centrifuge at 4000 rpm for 15 min |
| | 3. take out 150 pL and Dilute with 50 pL of 0.1% FA in water |
| | 4-Cap and vortex |
| | 5. inject 5 pL on LC-MS/MS |

CTC Autosampler

| Injection Volume | | | | Post valve |
|---|---|---|---|---|
| (pL) | 5 | Pre wash | Post wash | wash |
| Rinse Solution 1 | 50:50 (v:v) | 0 | 3 | 3 |
| | ACN:IPA | | | |
| Rinse Solution 2 | 75:25 (v:v) | 0 | 3 | 3 |
| | H2O:ACN | | | |
| Analytical column | Xbridge BEH C18 2.1 × 30 mm, 2.5 pm | | | |
| | (COL-060) | | | |
| Temperature (° C.) | 50 C. | | | |

Analytical pump

| | | | Flow rate | |
|---|---|---|---|---|
| Time (Min) | % A | % B | (pL/min) | A: |
| 0.00 | 95.0 | 5.0 | 500.0 | 0.1% Formic acid in water |
| 0.50 | 50.0 | 50.0 | 500.0 | B: |
| 1.80 | 5.0 | 95.0 | 500.0 | 0.1% Formic acid in IPA/ |
| | | | | ACN (1/4) |
| 2.80 | 5.0 | 95.0 | 500.0 | |
| 2.90 | 95.0 | 5.0 | 500.0 | |
| 3.50 | 95.0 | 5.0 | 500.0 | |

TSQ Quantum 2

| Ion Source | HESI II | | | |
|---|---|---|---|---|
| Polarity Mode | Positive | | | |
| Spray voltage | 3000 | | | |
| Vaporaizer | 500 | | | |
| Sheath Gas Pressure | 60 | | | |
| Ion sweep Gas | 1 | | | |
| Aux Gas Pressure | 5 | | | |
| Capillary | 350 | | | |
| Declustering Voltage | 0 | | | |
| S-Lens RF | SRM Table | | | |
| Collision Pressure | 1.5 | | | |
| Resolution Q1 | Unit | | | |
| Resolution Q3 | Unit | | | |
| Period | 1 | | | |
| Compound | Q1/Q3 | Dwell | CE | S Lens |
| Glyburide | 494.3/169.0 | 20 | 42 | 100 |
| Labetalol | 329.1/162.0 | 20 | 24 | 89 |
| KB-1801 | 476.15/84.0 | 20 | 32 | 132 |
| Potential | 652.16/84.0 | 20 | 32 | 132 |
| Glucuronide | | | | |
| metabolite | | | | |
| Potential | 652.16/176 | 20 | 18 | 132 |
| Glucuronide | | | | |
| metabolite | | | | |
| Potential | 652.16/260.0 | 20 | 32 | 132 |
| Glucuronide | | | | |
| metabolite | | | | |
| Time (min) | 3.5 min | | | |

A summary of the urine analytical method is provided in Table 73.

TABLE 73
3. Analytical Method Summary Urine
Study Summary

| | |
|---|---|
| Study Number | PK-2019-090 |
| Compound(s) | KB-1801 |
| Matrix | Rat Urine |
| Analysis Date (s) | 28 Mar. 2019 |
| LC/MS System | Quantum 1 |

Sample Preparation

| Extraction Method | Protein precipitation | | | | |
|---|---|---|---|---|---|
| Procedure | 1) 5 pL sample + 5 pL of 0.5% formic acid in water + 200 pL internal | | | | |
| | standard working solution (2 pM Glyburide/Labetalol in 0.1% formic acid | | | | |
| | in 0.5% Ammonium formate in Methanol/ACN) | | | | |
| | 2) Vortex and filter on a Protein P3 filter plate | | | | |
| | 3) Transfer 50 pL to another 2-mL deep well plate and dilute with 300 | | | | |
| | pL of (0.1% formic acid in water/0.1% formic acid in 0.5% Ammonium | | | | |
| | formate in 50/50 Methanol/ACN) (25/75) | | | | |
| | 4) Cap and vortex | | | | |
| | 5) inject 5 pL on LC-MS/MS | | | | |
| CTC Autosampler | | | | | |
| Injection Volume | | | | ost valve | |
| (pL) | 5 | Pre wash | Post wash P | was | h |
| Rinse Solution 1 | 50:50 (v:v) | 0 | 3 | 3 | |
| | ACN:IPA | | | | |
| Rinse Solution 2 | 75:25 (v:v) | 0 | 3 | 3 | |
| | H2O:ACI | | | | |
| Analytical column | Xbridge BEH C18 2.1 × 30 mm, 2.5 pm | | | | |
| | (COL-060) | | | | |
| Temperature (° C.) | 50 C. | | | | |

Analytical pump

| | | | Flow rate | |
|---|---|---|---|---|
| Time (Min) | % A | % B | (pL/min) | A: |
| 0.00 | 95.0 | 5.0 | 500.0 | 0.1% Formic acid in water |
| 0.50 | 50.0 | 50.0 | 500.0 | B: |
| 1.80 | 5.0 | 95.0 | 500.0 | 0.1% Formic acid in IPA/ACN |
| | | | | (1/4) |
| 2.80 | 5.0 | 95.0 | 500.0 | |
| 2.90 | 95.0 | 5.0 | 500.0 | |
| 3.50 | 95.0 | 5.0 | 500.0 | |

TSQ Quantum 1

| Ion Source | HESI II | | | |
|---|---|---|---|---|
| Polarity Mode | Positive | | | |
| Spray voltage | 3000 | | | |
| Vaporaizer | 400 | | | |
| Sheath Gas Pressure | 60 | | | |
| Ion sweep Gas | 1 | | | |
| Aux Gas Pressure | 5 | | | |
| Capillary | 380 | | | |
| Declustering | 0 | | | |
| S-Lens RF | SRM Table | | | |
| Collision Pressure | 1.5 | | | |
| Resolution Q1 | Unit | | | |
| Resolution Q3 | Unit | | | |
| Period | 1 | | | |
| Compound | Q1/Q3 | Dwell | CE | S Lens |
| Glyburide | 494.3/169.0 | 25 | 34 | 111 |
| Labetalol | 329.1/162.0 | 25 | 24 | 89 |
| KB-1801 | 476.15/84.0 | 25 | 32 | 134 |
| Potential | 652.16/84.0 | 25 | 32 | 132 |
| Potential | 652.16/176 | 25 | 18 | 132 |
| Potential | 652.16/260.0 | 25 | 32 | 132 |
| Time (min) | 3.5 min | | | |

The results show that during continuous intravenous infusion plasma PPTN concentrations reached steady state within approximately 24 hours and that PPTN concentrations were linearly dose-related. Plasma PPTN concentrations were reduced 10-fold to 30-fold within 24 hours after discontinuation of a 72-hour continuous infusion. PPTN was excreted in the urine at concentrations generally in excess of plasma concentrations and well above those known to inhibit P2Y14 receptor activation in vitro. PPTN/SBECD formulations were well tolerated by all rats; no adverse events or observations reported in-life, at necropsy, in blood clinical chemistry or cell count analyses.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A pharmaceutical formulation comprising a naphthoic acid derivative or salt thereof and an agent selected from the group consisting of α-tocopherol polyethylene glycol succinate (TPGS) and sulfobutyl ether beta-cyclodextrin (SBECD).

2. The pharmaceutical formulation of claim 1, wherein the naphthoic acid derivative is 4-((piperidin-4-yl)-phenyl)-(7-(4-(trifluoromethyl)-phenyl)-2-naphthoic acid (PPTN).

3. The pharmaceutical formulation of claim 1, wherein the naphthoic acid derivative salt is PPTN hydrochloride.

4. The pharmaceutical formulation of claim 1, wherein a pH of the pharmaceutical formulation is greater than 5.0.

5. The pharmaceutical formulation of claim 1, wherein the naphthoic acid derivative is present at ≥0.2 μg/ml.

6. The pharmaceutical formulation of claim 1, wherein the agent is TPGS and the pharmaceutical formulation further comprises saline or phosphate buffered saline (PBS).

7. The pharmaceutical formulation of claim 6, wherein the TPGS is present at less than 10%.

8. The pharmaceutical formulation of claim 6, wherein the pharmaceutical formulation further comprises DMSO.

9. The pharmaceutical formulation of claim 8, wherein the DMSO is present at less than 1%.

10. The pharmaceutical formulation of claim 1, wherein the agent is SBECD.

11. The pharmaceutical formulation of claim 10, wherein the pharmaceutical formulation further comprises saline or phosphate buffered saline (PBS).

12. The pharmaceutical formulation of claim 10, wherein the SBECD is present at less than 20%.

13. The pharmaceutical formulation of claim 10, wherein the SBECD is present at less than 10%.

14. The pharmaceutical formulation of claim 10, wherein the SBECD is present at less than 5%.

15. A method for treating a renal disorder, the method comprising providing to a subject a pharmaceutical formulation comprising a naphthoic acid derivative or salt thereof and an agent selected from the group consisting of α-tocopherol polyethylene glycol succinate (TPGS) and sulfobutyl ether beta-cyclodextrin (SBECD).

16. The method of claim 15, wherein the naphthoic acid derivative is 4-((piperidin-4-yl)-phenyl)-(7-(4-(trifluoromethyl)-phenyl)-2-naphthoic acid (PPTN).

17. The method of claim 15, wherein the naphthoic acid derivative salt is PPTN hydrochloride.

18. The method of claim 15, wherein a pH of the pharmaceutical formulation is greater than 5.0.

19. The method of claim 15, wherein the agent is TPGS and the pharmaceutical formulation further comprises saline or phosphate buffered saline (PBS).

20. The method of claim of claim 19, wherein the TPGS is present at less than 10%.

21-27. (canceled)
